(19) United States
(12) Patent Application Publication
(10) Pub. No.: US 2021/0290313 A1
(43) Pub. Date: Sep. 23, 2021

(54) COMPUTER-ASSISTED TRACKING SYSTEM USING ULTRASOUND

(71) Applicant: ORTHOSOFT ULC, Montreal (CA)

(72) Inventors: Victor CERDA-CARVAJAL, Montreal (CA); Antoine BAUTIN, Ponet Saint Auban (FR); Gregory STRUBEL, Manosque (FR); Joseph MADIER-VIGNEUX, Montreal (CA); Pierre-François BEAUCHEMIN, Montreal (CA); Louis-Philippe AMIOT, Montreal (CA); Guillaume BOIVIN, Montreal (CA); Bassam JABBOUR, Saint-Laurent (CA); Jean THURIET, Montreal (CA)

(21) Appl. No.: 17/206,552

(22) Filed: Mar. 19, 2021

Related U.S. Application Data
(60) Provisional application No. 62/991,707, filed on Mar. 19, 2020.

(51) Int. Cl. A61B 34/20; A61B 8/00; A61B 8/08

(52) U.S. Cl. CPC A61B 34/20; A61B 8/4488; A61B 8/08; A61B 8/4281; A61B 2034/2065; A61B 2034/2063; A61B 2034/2059; A61B 2034/2057; A61B 2034/2068

(57) ABSTRACT

There is described an ultrasound tracking system for tracking a position and orientation of an anatomical feature in computer-assisted surgery. The system generally has: an ultrasound imaging system having a phased-array ultrasound probe unit for emitting ultrasound signals successively towards different portions of said anatomical feature, measuring echo signals returning from said portions of said anatomical feature and generating respective imaged echo datasets; a coordinate tracking system tracking coordinates of said ultrasound phased array probe unit during said measuring, and generating corresponding coordinate datasets; and a controller being communicatively coupled to said ultrasound imaging system and said coordinate tracking system, said controller performing the steps of: registering said imaged echo datasets in a common coordinate system based on said coordinate datasets; and tracking said position and orientation of said anatomical feature based on said registering.

### TECHNICAL FIELD

The present disclosure relates to the field of computer-assisted surgery, and more specifically, to anatomical feature tracking and positioning in computer-assisted surgery (CAS) systems.

### BACKGROUND

Computer-assisted surgery (CAS) makes use of references fixed to the patient using pins inserted into the bones of the limbs or the pelvis. These pins, inserted into the bones before or during the surgery, are of different diametrical sizes and can cause pain after the surgery. They are an extra step to the surgery, exclusively because of the navigation system. Also, the insertions of the pins into the bone may cause weaknesses of the bone that can then more easily be fractured. Some cases, involving osteoporotic bones may also cause anchoring instability due to the lack of density of the bone. Infections may also occur as for any entry point at surgery.

Furthermore, the length of the pins is sometimes obtrusive to the surgeon who may cut them to a length better adapted to his movement during the surgery. The cut is also perceived as a nuisance; its end may be sharp and hazardous to the personnel working around the surgery table.

Consequently, wearable trackers have been developed, so as to minimize the invasive nature of computer-assisted tracking devices. However, the wearable trackers may occasionally lack precision. There thus remains room for improvement.

### SUMMARY

In accordance with a first aspect of the present disclosure, there is provided an ultrasound tracking system for tracking a position and orientation of an anatomical feature in computer-assisted surgery, the ultrasound tracking system comprising: an ultrasound imaging system having a phased-array ultrasound probe unit being adapted for emitting ultrasound signals successively towards different portions of said anatomical feature, measuring echo signals returning from said portions of said anatomical feature and generating respective imaged echo datasets; a coordinate tracking system tracking coordinates of said ultrasound phased array probe unit during said measuring, and generating corresponding coordinate datasets; and a controller being communicatively coupled to said ultrasound imaging system and said coordinate tracking system, said controller having a processor and a memory having stored thereon instructions that when executed by said processor perform the steps of: registering said imaged echo datasets in a common coordinate system based on said coordinate datasets; and tracking said position and orientation of said anatomical feature based on said registering.

In accordance with a second aspect of the present disclosure, there is provided a method for tracking a position and orientation of an anatomical feature in computer-assisted surgery, the method comprising: emitting phased-array ultrasound signals towards different portions of said anatomical feature, measuring echo signals returning from said portions of said anatomical feature and generating respective imaged echo datasets while tracking coordinates of said ultrasound imaging system, and generating corresponding coordinate datasets; and a controller performing the steps of: registering said imaged echo datasets in a common coordinate system based on said coordinate datasets; and tracking said position and orientation of said anatomical feature based on said registering.

In accordance with a third aspect of the present disclosure, there is provided a wearable element for use in computer-assisted surgery involving ultrasound tracking of an anatomical feature of a patient, the wearable element comprising: a garment to be worn by the patient; and an ultrasound imaging interface covering at least a portion of the garment, the ultrasound imaging interface being made of a solid acoustically transmissive material and having one or more surgery openings defined therein allowing access to the anatomical feature. In some embodiments, one or more ultrasound probe units can be embedded into the ultrasound imaging interface. In some embodiments, the garment can be provided in the form of a compression shirt, a compression sleeve and the like.

In accordance with a fourth aspect of the present disclosure, there is provided an ultrasound tracking device for use with a position sensing system to register position and orientation in computer-assisted surgery, the ultrasound tracking device comprising: a wearable holder adapted to be secured to an anatomic feature; at least two ultrasonic probe units supported by the wearable holder and adapted to emit signals to image part of the anatomic feature; at least one reference tracker supported by the wearable holder; and a mechanical member projecting from a remainder of the ultrasound tracking device and increasing an axial footprint of the ultrasound tracking device. In some embodiments, the at least two ultrasonic probe units are axially spaced-apart from one another along an anatomical axis of the anatomical feature.

In accordance with a fifth aspect of the present disclosure, there is provided a set of ultrasound tracking devices for use with a position sensing system to register position and orientation in computer-assisted surgery, each of the ultrasound tracking device comprising: a wearable holder adapted to be secured to an anatomic feature, and at least two ultrasonic probe units supported by the wearable holder and adapted to emit signals to image part of the anatomic feature; at least one reference tracker supported by one of the wearable holders; and a linkage between the set of ultrasound tracking devices, the linkage having a rotational joint and a sensor for determining an angular value variation in the rotational joint. In some embodiments, the ultrasound tracking devices are axially spaced-apart from one another along an anatomical axis of the anatomical feature.

In accordance with a sixth aspect of the present disclosure, there is provided an ultrasound tracking system for tracking a position of the ultrasound tracking device with respect to an extremity of an anatomical feature in computer-assisted surgery, the ultrasound tracking system comprising: at least an ultrasound probe unit fixedly mounted relative to the anatomical feature, the ultrasound probe unit being adapted for emitting an ultrasound signal within said anatomical feature, at least a portion of the ultrasound signal being guided away from the ultrasound probe unit and along an anatomical axis of the anatomical feature towards and the extremity thereof, the ultrasound probe unit detecting at least a reflected portion of the ultrasound signal being guided from the extremity of the anatomical feature and back towards the ultrasound probe unit; a controller being communicatively coupled to said ultrasound probe unit, said controller having a processor and a memory having stored thereon instructions that when executed by said processor perform the steps of: determining an axial position of the ultrasound probe unit relative to the extremity of the anatomical feature based on an ultrasound speed value indicative of a speed at which the portion of the ultrasound signal travels along the anatomical feature and on a time duration indicative of a time duration elapsed between the emitting and the detecting. In some embodiments, the ultrasound speed value is measured in situ based on measurements performed by at least two ultrasound probe units axially spaced-apart from one another along the anatomical axis.

In this specification, the term “reference marker” is intended to mean an active or passive marker, such as an emitter, a transmitter or a reflector.

### BRIEF DESCRIPTION OF THE DRAWINGS

Further features and advantages of the present disclosure will become apparent from the following detailed description, taken in combination with the appended drawings, in which:

FIG. 1 is a schematic view of a computer-assisted ultrasound tracking system, in accordance with one or more embodiments;

FIG. 2 is a cross-sectional view of a limb featuring an elongated bone, such as a femur, with an ultrasound tracking device onto the limb, in accordance with one or more embodiments;

FIG. 3 is a perspective view of another example of an ultrasound tracking device, incorporating an open cuff body, in accordance with one or more embodiments;

FIG. 3A is a graph showing exemplary imaged echo datasets generated by the ultrasound tracking device of FIG. 3, in accordance with one or more embodiments;

FIG. 3B is a graph showing a sectional mapping of an anatomical feature imaged by the ultrasound tracking device of FIG. 3, in accordance with one or more embodiments;

FIG. 4 is a schematic view of a pair of ultrasound tracking devices with mechanical members, in accordance with one or more embodiments;

FIG. 5 is a schematic view of a pair of ultrasound tracking devices with a linkage therebetween, in accordance with one or more embodiments;

FIG. 5A is a schematic view of a pair of ultrasound tracking devices with rigid connection to a referenced structure, in accordance with one or more embodiments;

FIG. 5B is a schematic view of a pair of ultrasound tracking devices with rigid connection to the referenced structure of FIG. 5A, in accordance with one or more embodiments;

FIG. 6 is a perspective view of a patient using the pair of ultrasound tracking devices with rigid connection as in FIG. 5B, in accordance with one or more embodiments;

FIG. 7 is a schematic view of a pair of ultrasound tracking devices in a phased array system, in accordance with one or more embodiments;

FIG. 8 is a schematic view of an example of an ultrasound tracking system for use in computer-assisted surgery, shown with an ultrasound imaging system, a coordinate tracking system and a controller, in accordance with one or more embodiments;

FIG. 8A is a perspective view of ultrasound probe units of the ultrasound tracking system of FIG. 8, shown at respective coordinates during measurements of echo signals returning from portions of an anatomical feature, in accordance with one or more embodiments;

FIG. 8B is a graph showing an example of an imaged echo dataset associated to a first measured echo signal plotted in a first coordinate system, in accordance with one or more embodiments;

FIG. 8C is a graph showing an example of an imaged echo dataset associated to a second measured echo signal plotted in a second coordinate system, in accordance with one or more embodiments;

FIG. 8D is a graph showing the imaged echo datasets of FIGS. 8B and 8C registered in a common coordinate system based on tracked coordinates of the ultrasound probe units during the measurements, in accordance with one or more embodiments;

FIG. 9 is a schematic view of an example of a computing device of the controller of FIG. 8, in accordance with one or more embodiments;

FIG. 10 is a flow chart of an example of a method for tracking a position and orientation of an anatomical feature in computer-assisted surgery using an ultrasound tracking system, in accordance with one or more embodiments;

FIG. 11 is a perspective view of another example of an ultrasound tracking system, with an optical coordinate tracking system, in accordance with one or more embodiments;

FIG. 12 is a perspective view of another example of an ultrasound tracking system, with a mechanical coordinate tracking system, in accordance with one or more embodiments;

FIG. 12A is a sectional view of the ultrasound tracking system of FIG. 12, taken along section 12A-12A of FIG. 12;

FIG. 13 is a perspective view of an example of a wearable element made of an acoustically transmissive material and having surgery openings to be worn of a patient's thoraco-lumbar region, in accordance with one or more embodiments; and

FIG. 14 is a perspective view of another example of a wearable element to be worn on a patient's shoulder, in accordance with one or more embodiments.

Many further features and combinations thereof concerning the present improvements to those skilled in the art following a reading of the instant disclosure.

### DETAILED DESCRIPTION

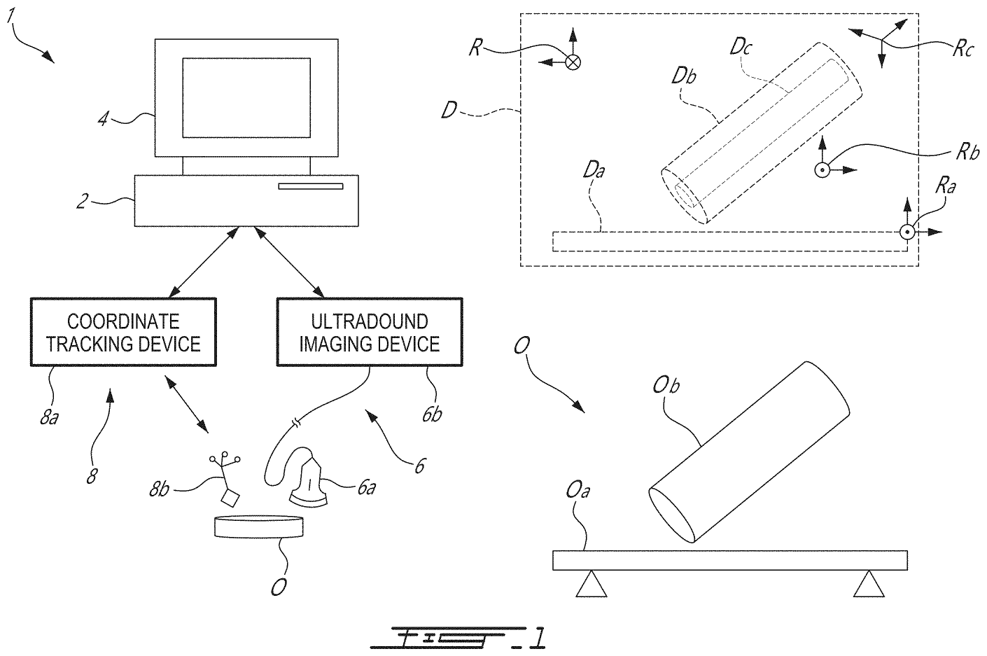

Referring to the drawings and more particularly to FIG. 1, a computer-assisted (CAS) tracking system in accordance with one aspect of the present disclosure is shown at 1. The CAS tracking system 1 is provided for performing CAS tracking of certain objects O including, for example, anatomical features (such as bones and soft tissue), spatial references (such as marker-like devices and, in some implementations, anatomical landmarks), and tools, as described hereinafter. It is also contemplated that the CAS tracking system 1 may also be used to track a patient, in some cases indirectly via a wearable object O. The CAS tracking system 1 may have a controller 2. As shown, the controller 2 may be part of a computer, or may be implemented in the form of a personal computer, a laptop, a tablet, a server, etc., that may be dedicated to CAS tracking and surgical flow assistance. The controller 2 may be configured for executing computer-readable program instructions suitable for the processing of datasets D related to CAS tracking. The program instructions may originate from a medium either remote from the controller 2 or directly connected thereto. Among possible implementations, the program instructions may be stored on a non-transitory computer-readable medium which may be communicatively coupled to the controller 2.

As the CAS tracking system 1 operates, the controller 2 may receive, generate and transfer at least some of the datasets D associated to the objects O, which may be of various types of information including spatial (e.g., position, orientation), surfacic and volumetric. The controller 2 may also be used to derive information from such datasets D, which may include modifying and/or combining some such datasets D. The controller 2 may be capable of parsing or otherwise registering any such dataset D so as to interpret it in terms of an object-agnostic coordinate system, which may be referred to as a reference coordinate system R, a frame of reference, a referential system, etc. It should be noted that the controller 2 may relate some of the datasets D to one another. For example, a first dataset Dₐ and a second dataset D_{b} may respectively represent the position and orientation of a first object Oₐ and of a second object O_{b} according to corresponding first Rₐ and second R_{b} coordinate systems, or in a common global coordinate system R. The controller 2 may be configured to parse the second dataset D_{b} so as to interpret it as if it were defined according to another coordinate system, for example the first coordinate system Rₐ or the reference coordinate system R. In some embodiments, the first dataset Dₐ may be interpreted in a first coordinate system Rₐ and may be modified by registering it into another coordinate system such as the reference coordinate system R, for instance. In other words, the first and second datasets may be registered to one another in a common coordinate system, i.e., the reference coordinate system R.

The CAS tracking system 1 of FIG. 1 provides, as an output, computer-assisted surgery guidance to the operator by timely communication of the above datasets D and/or information, referred to henceforth as navigation data, in pre-operatively planning, peri-operatively, or during the surgical procedure, i.e., intra-operatively. The CAS tracking system 1 may comprise various types of interfaces for the navigation data to be suitably communicated to the operator, for instance via the GUI 4 shown as part of the controller 2. In some embodiments, the tracked anatomical features can be displayed in real time on the GUI 4 during the computer-assisted surgery. The interfaces of the CAS tracking system 1 may be monitors, displays and/or screens including mounted devices, wireless portable devices (e.g., phones, tablets), head-up displays (HUD), augmented-reality devices such as the Hololens®, Microsoft® (Redmond, Wash.), audio guidance devices and haptic feedback devices, mouse, keyboard, foot pedal, and any combination thereof, among many other possibilities. A single-device interface is also contemplated.

In terms of input, the CAS tracking system 1 may have access to some of datasets D in the form of digital models for the various objects O, such as anatomical models including, but not limited to, arm model(s), bone model(s), artery model(s), vein model(s), nerve model(s) and model(s) of any other anatomical feature(s) of interest. Such anatomical models may consist in datasets containing information relating to surfacic and/or volumetric characteristics of corresponding anatomical features, such as a bone. The anatomical models may be patient-specific, and may have been obtained pre-operatively or peri-operatively, using various imaging modalities. For example, the anatomical models may have been generated from magnetic resonance imagery, radiography in its various forms, ultrasound imaging, to name a few examples. As the case may be, a dataset D_{c} corresponding to a bone model may be defined according to a coordinate system R_{c} consistent with imaging conventions, e.g., with the X, Y and Z axes corresponding to the anterior, left lateral and cranial directions upon the patient lying supine. In some embodiments, the bone models may be obtained from a bone atlas or other suitable source based on factors such as gender, race, age, etc, for example as described in U.S. Pat. Nos. 8,884,618, and 10,130,478, the contents of both of which being incorporated herein by reference. In some such embodiments, the bone models may be digitally fitted to patient-specific data, such as, for instance, partial yet strategically selected data points, such as for joint surfaces of a bone, while a bone shaft may be taken from a bone atlas, for example. Such processing of the models may be carried out remotely or locally (e.g., via the controller 2). Likewise, storage of the bone models and/or models of any other anatomical features may be implemented remotely or locally (i.e., via a computer-readable memory).

Moreover, the CAS tracking system 1 has an ultrasound imaging system 6 configured to generate some of the datasets D. The ultrasound imaging system 6 includes at least one ultrasound probe unit 6a provided for producing signals indicative of characteristics pertaining to the objects O. The resulting signals may be communicated from an ultrasound imaging device 6b to the controller 2 to be processed into corresponding datasets D. Alternatively, the signals may be processed by device-specific processing units, such that the corresponding datasets D may be received by the controller 2 instead of the signals. The ultrasound imaging system 6 may be said to be modular as it can include a plurality of ultrasound probe unit(s) 6a and/or ultrasound imaging device(s) 6b.

Further, the CAS tracking system 1 may be configured such that the outputting of at least some of the navigation data from the controller 2 is timed with inputting of at least some of the datasets D into the controller 2. The CAS tracking system 1 may thus be said to provide the navigation data in real time or near real time.

In accordance with some embodiments, a coordinate tracking system 8 is provided as part of the CAS tracking system 1. The coordinate tracking system 8 may include one or more coordinate tracking devices 8a including, for instance, a camera that tracks marker reference(s) 8b. The coordinate tracking system 8 can use either active or passive spatial references as markers of position and/or orientation. For example, as is known in the art, the coordinate tracking system 8 may optically see and recognize retroreflective devices as reference markers, so as to track objects, for example tools and limbs, in six degrees of freedom (DOFs), namely in position and orientation along the X, Y and Z axes. Thus, the orientation and position of the limb in space can be determined using the information obtained from the spatial references, resulting in a corresponding dataset (for example, the dataset D_{b}) that is defined according to a corresponding coordinate system (for example the coordinate system R_{b}), which may in some cases be inherent to a reference marker or to the ultrasound probe unit 6a used therewith. The coordinate tracking system 8 may also include a dedicated computing device used to condition, digitize and/or otherwise process the signal produced by the camera. The coordinate tracking device 8a may be a 3D depth camera as a possibility (e.g., a Kinect™), that may not require passive spatial references as markers of position and/or orientation. Other 3D cameras can be used in other embodiments. For instance, the coordinate tracking device 8a may include conventional two-dimensional camera(s) (operated in mono- or stereo-vision configuration) operated with a shape recognition module which identifies, locates and processes two-dimensional identifiers (e.g., QR codes) as imaged in the two-dimensional images generated by the two-dimensional camera(s). In these embodiments, the shape recognition module can evaluate the distortion of the two-dimensional identifiers in the two-dimensional images (e.g., a square identifier becoming trapezoidal when bent) to retrieve three-dimensional model(s) of the two-dimensional identifiers and/or of the underlying anatomical feature.

In some embodiments, the ultrasound imaging system 6 is used to produce a signal indicative of at least one spatial and/or dimensional characteristic relating to biological tissue. According to conventional ultrasound-based detection principles, which are typical to conventional ultrasound probe units, an ultrasound emitter may be used to cast a sound wave and, upon an object being located within range of the ultrasound emitter, an echo of the sound wave is cast back to be sensed by an ultrasound sensor. In some embodiments, the ultrasound emitter and the ultrasound sensor may be separate from one another. However, in some other embodiments, the ultrasound emitter and the ultrasound sensor may be combined to one another in an ultrasound transducer performing both the ultrasound emission and the sensing functions. The echo may materialize upon the sound wave travelling through a first medium, such as skin, reaching a second medium of greater density compared to that of the first medium, such as a bone. As the speeds at which the sound waves may travel through various media depend on the respective physical properties of such media, characteristics of the echo (e.g., time elapsed between emission of the sound wave and the sensing of the echo, intensity of the echo relative to that of the sound wave, etc.) may be used to derive certain characteristics of the media through which the echo has travelled. In some embodiments, the functions of both the ultrasound emitter and the ultrasound sensor are performed by one or more ultrasound transducer transducers. In some embodiments, the ultrasound transducer may have one or more piezoelectric crystals emitting ultrasound signals based on corresponding electrical signals, and/or generating electrical signals based on received ultrasound signals. Any suitable type of ultrasound transducers can be used including, but not limited to, piezoelectric polymer-based ultrasound transducers such as poly(vinylidene fluoride)-based ultrasound transducers, capacitive ultrasound transducers, microelectromechanical systems (MEMS) based ultrasound transducers and the like.

Per the present disclosure, namely, in the exemplary case of orthopedic surgery for instance, the ultrasound imaging system 6 may be configured to produce a signal indicative of a detailed spatial relationship between the ultrasound probe unit 6a and a limb (which may be one being tracked by the coordinate tracking system 8), and also between constituents of the limb such as soft tissue (e.g., skin, flesh, muscle, ligament) and bone. Resulting datasets may include measurements of a distance between contours associated to the limb, such as an epithelial contour associated to skin and a periosteal contour associated to the bone. The resulting datasets may also include measurements of thicknesses, surfaces, volumes, medium density and the like. Advantageously, updated signal production via the ultrasound imaging system 6 and ad hoc, quasi-real-time processing may produce datasets which take into account movement and/or deformation of one or more of the constituents of the limb. The ultrasound imaging system 6 may also include a dedicated computing device configured for conditioning and/or digitizing the signal.

In some implementations, the ultrasound imaging system 6 may be suitable for producing a signal indicative of surfacic, volumetric and even mechanical properties of the objects O to be tracked by the CAS tracking system 1. This may be achieved, for instance, by way of a multi-planar ultrasound system capable of operating simultaneously along multiple notional planes that are spaced and/or angled relative to one another, coupled to a suitably configured controller 2. Further, it is contemplated that other types of imaging systems, such as an optical coherence tomography (OCT) system, may be used in combination with the ultrasound imaging system 6. The type of additional imaging system may be selected, and combined with other type(s) as the case may be, to attain certain performance requirements in terms of effective range, effective depth, signal-to-noise ratio, signal acquisition frequency, contrast resolution and scale, spatial resolution, etc., among other possibilities. In some embodiments, partially exposed bone structures may be captured and/or referenced by the additional imaging system at any time before, during or after the surgery. Specifications of such imaging systems may thus be adapted, to some degree, based on requirements derived from typical characteristics of the objects O to be tracked.

As will be described in view of the above, a precise tracking of bone may be achieved using the CAS tracking system 1, regardless of certain materials, such as soft tissue, being overlaid thereon.

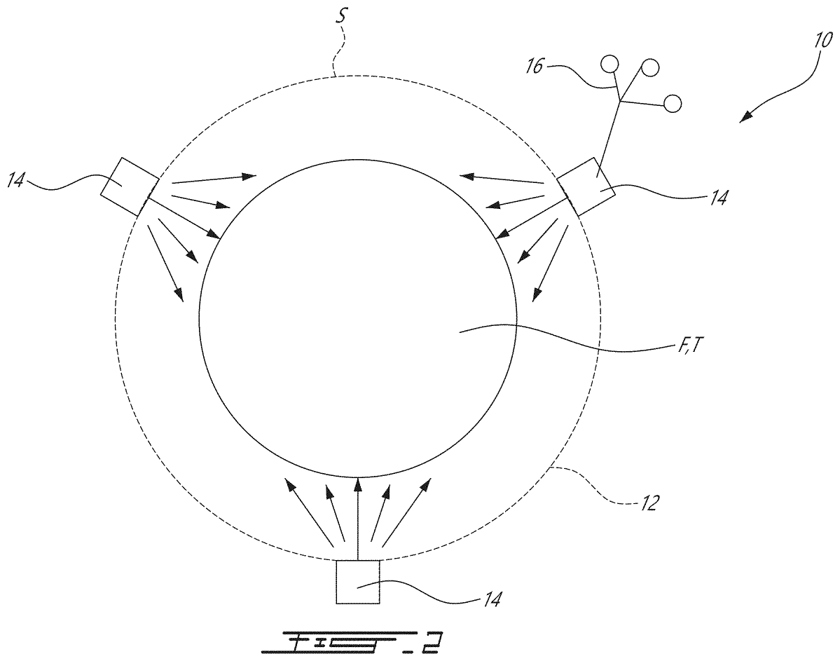

The CAS tracking system 1, and specifically the coordinate tracking system 8, may be well suited to track ultrasound tracking devices 10 shown in FIG. 2.

According to an embodiment, an ultrasound tracking device 10 is of the type that attaches to a limb of a patient, and is used to track an axis of the limb. For this purpose, the ultrasound tracking device 10 has a wearable holder 12, ultrasound probe units 14, and may have another trackable reference 16.

The wearable holder 12 is of the type that is mounted about the outer-skin surface S (a.k.a., exposed skin, epidermis, external soft tissue, etc.) of an anatomical feature, such as but not limited to a thigh with femur F or a shank with tibia T of a patient. The wearable holder 12 and the CAS tracking system using it, as will be described herein and as an example the ultrasound imaging system 6 of FIG. 1, may therefore be used to determine the position and/or orientation of femur

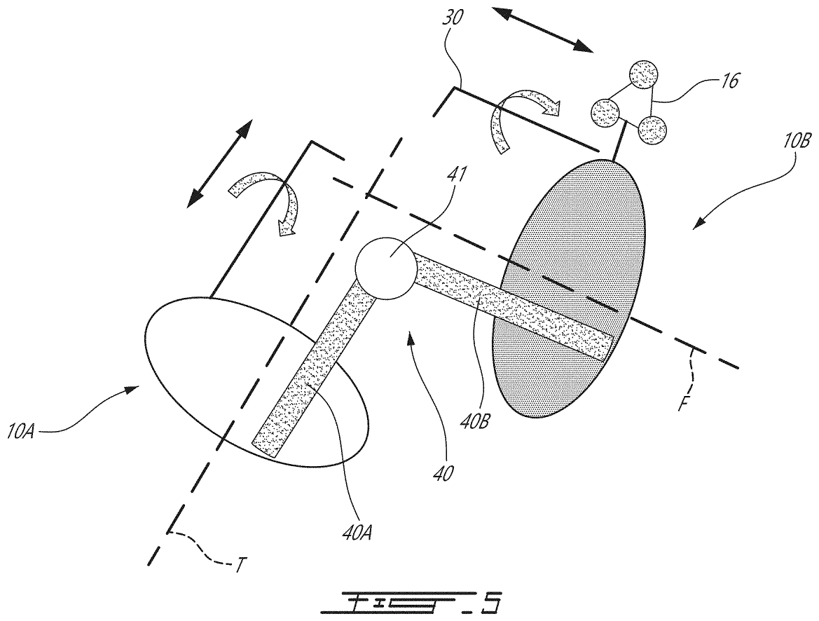

F and tibia T in FIGS. 4 to 7, but also other anatomical features of arms (humerus, forearm, etc.), other joints (e.g., elbow, hip, shoulder, etc.), nerves, arteries, veins, and the like. In an embodiment featuring the ultrasound tracking device 10 and CAS tracking system, the bones may be largely subcutaneous, in that a majority thereof is disposed beneath, and thus substantially underlies, the outer-skin surface S of the anatomical feature in question. In certain embodiments, the bone may thus be said to be substantially unexposed. However, it is to be understood that one or more portions of the bones may be exposed during surgery, for example as a result of one or more incision(s) made as part of the surgical technique being employed. Accordingly, while portions of the bone may be exposed during surgery with the anatomical feature within the wearable holder 12, the bone will otherwise remain substantially subcutaneous.

While the bone may be described herein as “underlying” the outer-skin surface S, it is to be understood that this does not exclude the possibility that certain portions of the bone may at least partially exposed during surgery (e.g. by incisions, etc.) nor does this require or imply that the entirety of the bone must necessarily be unexposed and subcutaneous at all times, for instance in the case of laparoscopic procedures.

The ultrasound tracking device 10 including the wearable holder 12 is configured to be grossly secured to the anatomical feature against which it is mounted in such a way that there is a tolerable movement between the holder 12 and the anatomical feature. Algorithms can detect and compensate for movement using ultrasound processing combined with the optical tracking system. The position and the orientation of the holder 12 may also be trackable through space by the CAS tracking system, whereby a tracking of the anatomical feature can be derived from a tracking of the ultrasound tracking device 10. The ultrasound tracking device 10 is therefore a non-invasive tool to be used to track the position and the orientation, and thus the movement, of the bone through space before, during or after the computer-assisted surgery, for instance relative to a global referential system.

The wearable holder 12 of the ultrasound tracking device 10 can take different forms to accomplish such functionality. In the depicted embodiment, the wearable holder 12 is in the form a belt, ring, vest or strap that is mounted to an anatomical feature (e.g., a leg and the underlying femur or tibia, etc.) of the patient to be in fixed relative relationship with the bone. In an alternate embodiment, the wearable holder 12 is in the form a tight-fitting sleeve that is mounted to an anatomical feature of the patient to be in fixed relative relationship with the bone. Similarly, the wearable holder 12 is mountable about other limbs, appendages, or other anatomical features of the patient having a bone to be tracked. The wearable holder 12 may essentially be a pressurized band around the limb to enhance contact. It is also considered to use a gel conforming pad to couple the holder 12 to the skin, as a possibility. Traditional coupling gel can also be used. In some embodiments, coupling gel of typical formulations as well as biocompatible gel (e.g., in vivo biocompatible or in vivo bioexcretable) can be used. The gel conforming pad may include acoustically transmissive material which can help the transmission of the ultrasound signals and returning echo signals thereacross. In another embodiment, the wearable holder 12 is in the form of a boot, a glove or a corset (in the thoraco-lumbar region). The wearable holder 12 may be annular and arranged to be at an axial location corresponding to a slice of the bone, to which the bone axis is normal, in a first scenario. However, it is also considered to have the holder 12 angled, in such a way that the bone axis is not normal to a plane passing though the holder 12. In doing so, the ultrasound tracking device 10 may produce a greater axial coverage of bone surface than for the first scenario. Other embodiments can also include independently placed sensors that are disposed in a non-planar but relevant scanning positions to obtain usable datasets.

Ultrasound probe units 14 are secured to the wearable holder 12. In an embodiment, the ultrasound probe units 14 include one or more transducers that emit an ultrasound wave and measure the time it takes for the wave to echo off of a hard surface (such as bone) and return to the face(s) of the transducer(s). In order to self-calibrate for the patient's individual speed of sound, some transducers are positioned very accurately relative to others and as one emits waves, others listen and can compute the speed of sound based on well-known relative geometric positioning. Using the known speed of the ultrasound wave travelling through a bodily media, the time measurement is translated into a distance measurement between the ultrasound probe 14 and the bone located below the outer-skin surface S. The transducers in the probe units 14 may be single-element or multi-element transducers, or a combination of both. For example, the probe units 14 may have multiple elements arranged in a phased array, i.e., phased-array ultrasound probe units 14, having the capacity of performing multi-element wave generation for sound wave direction control and signal reconstruction. In some embodiments, the phased-array ultrasound probe unit 14 has a single ultrasound transducer operating in a phased-array arrangement. When sensors are not rigidly linked to others, the relative position can be found with self-location algorithms. Therefore, the probe units 14 used in the manner shown in FIG. 2 produce signals allowing local image reconstruction of the bone. The phased-array ultrasound probe units 14 are configured to emit ultrasound signals successively towards different portions of the anatomical features. In some embodiments, the ultrasound signals may be successively steered from one portion to another. Alternatively or additionally, the ultrasound signals may be successively focused on the different portions of the anatomical feature. In another embodiment, the ultrasound probe units 14 are ultrasound devices integrated into the ultrasound tracking device 10. The measurement is done by either triggering it manually, or automatically. In one embodiment, the measurement is repeated at regular intervals. The measurements are constantly being transferred to the ultrasound tracking system of the CAS tracking system (FIG. 1), for the position and orientation of the bone in space may be updated. In an embodiment, a reference marker 16 may be part of the ultrasound tracking device 10. These reference markers 16 may be active or passive, optical (including fiber optic Bragg grating), RF, electro-magnetic, inertial or even ultrasound. In the figures, optical reflective reference markers 16 are illustrated, in the form of three tokens or spheres, for illustrative purposes. The reference markers 16 are recognized by the position sensing system by their distinct geometries, such that a CAS tracking system such as the one described with reference to FIG. 1, can determine the position and orientation of the ultrasound tracking devices 10 in space using the reference markers. The tracking of the ultrasound tracking devices 10 in space, combined to the image reconstruction data from the ultrasound probe units 14, is used to track the anatomical features, such as the axes of the femur F and tibia T. For example, the image reconstruction from the signals of the ultrasound tracking device(s) 10 may be used in conjunction with the bone models in the CAS tracking system to match or register the reconstructed image from ultrasound with the 3D bone models in the CAS tracking system, and hence position and orient the bones in the 3D space, i.e., the coordinate system. The registration may be performed automatically by the CAS tracking system.

FIG. 2 is a cross-sectional view of a limb, such as a thigh, a lower leg, or any other bone having an elongated form. The ultrasound probe units 14 are positioned around the bone on the outer-skin surface S via the wearable holder 12. The ultrasound probe units 14 are therefore distributed around the limb by the wearable holder 12. If the reference marker 16 is of the optical type, than the reference marker 16 must be in the line of sight of the position sensing system to be tracked. Other reference markers 16 may be used to increase a range of visibility of the ultrasound tracking device 10. Again, it is contemplated to angle the wearable holder 12 relative to the limb to increase axial surface coverage and reconstruct a bone surface having a greater axial span.

A set of two or more ultrasound probe units 14 may be needed to determine the anatomical axis, as illustrated in FIG. 2. The wearable holder 12 surrounds the limb of the patient. The wearable holder 12 is positioned such that there are pairs of probe units 14 facing each other. The anatomical axis of the bone may be determined by locating the middle point between a pair of probe units 14 and forming a line from these points along the bone. Moreover, the readings from the probe units 14 may be used to perform a 3D image reconstruction of the bone, by the processor of the CAS tracking system, and then identify a center of the bone segment, the anatomical axis passing through the center or being positioned relative to the center. The position of the wearable holder 12 in space may then be determined using the reference marker 16. Therefore, in an embodiment, one or more ultrasound probe units 14 are needed to determine the anatomical axis of a limb, if the reading from an ultrasound probe 14 provides a position from which more than one point on a line can be determined. It is also considered to position a pair of wearable holders 12 on a same bone, with an interconnection between them for example (or using the bone as being the “rigid connection” between the wearable holders 12). A single reference marker 16 could be shared in such a scenario, with the combination of the wearable holders 12 providing a further increase in axial surface coverage. In some embodiments, each wearable holder 12 may have a single ultrasound probe 14 equipped with an array of transducers operated in a phased array arrangement to determine the middle point, the cross-sectional shape, and/or an anatomical axis of the tracked bone.

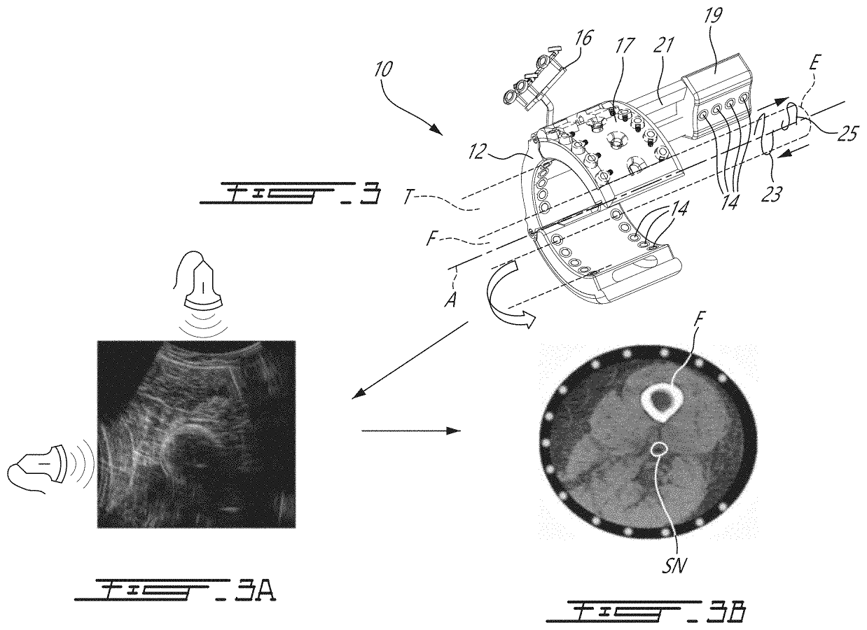

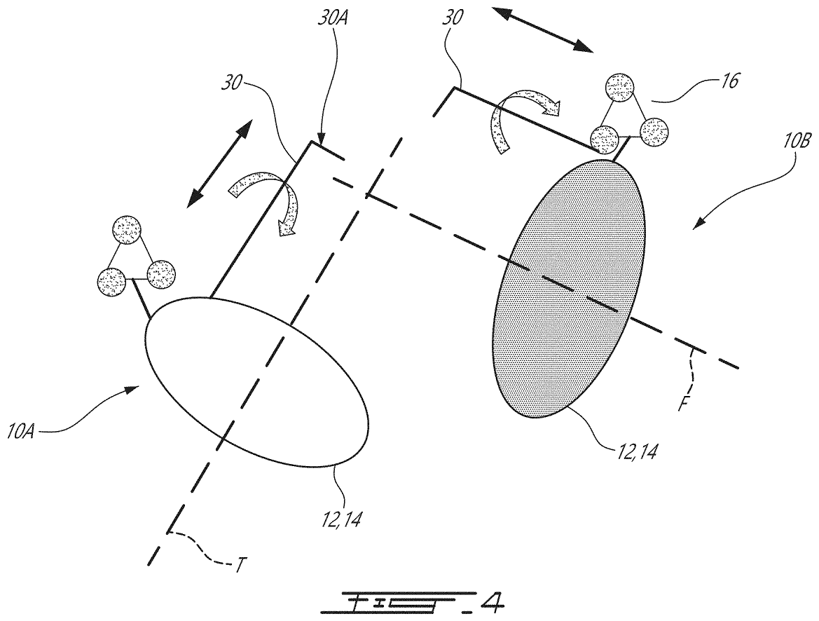

FIG. 3 shows another example of an ultrasound tracking device 10. As depicted in this embodiment, the ultrasound tracking device 10 is of the type that can be slid or otherwise installed onto a limb of a patient, and is used to track an anatomical axis A of the limb. For this purpose, the ultrasound tracking device 10 has a wearable holder 12, which in this case is provided in the form of a cuff, and ultrasound probe units 14 mounted to the wearable holder 12. Moreover, the ultrasound tracking device 10 may have a trackable reference 16 to track the position of the wearable holder 12 as it is moved prior to, during or after surgery.

In this specific embodiment, the wearable holder 12 has an open cuff body 17 being wrappable about patient's thigh T for the ultrasound imaging of the femur F, for instance. The wearable holder 12 also has elongated member 19 extending axially from a circumference of the open cuff body 17, in a manner parallel to the anatomical axis A, for example. As shown, both the open cuff body 17 and the elongated member 19 have respective ultrasound probe units 14 that are embedded therein. The ultrasound probe units 14 are radially inwardly oriented. In this embodiment, the open cuff body 17 has two axially spaced-apart arrays of circumferentially distributed ultrasound probe units 14, hence providing axial coverage. However, in some other embodiments, only one or more than two of these arrays can be provided. Similarly, more than one array of axially distributed ultrasound probe units 14 can be embedded in the elongated member 19 in some other embodiments. As can be appreciated, each of the ultrasound probe units 14 can be used either as an ultrasound transmitter or as an ultrasound receiver, or both, depending on the embodiment. In some embodiments, the wearable holder 12 can have an adjustment mechanism 21 allowing to adjust, e.g., increase or decrease, the distance between the ultrasound probe units 14 of the elongated member 19 and the ultrasound probe units 14 of the open cuff body 17. The tuning mechanism 21 can be telescopic in some embodiments, and it can be encoded to monitor the distance between the open cuff body 17 and the elongated member 19.

As best shown in FIG. 3A, imaged echo data sets generated by the ultrasound probe units 14 of the open cuff body 17 can be plotted in a common referential coordinate system thanks to the tracking of the trackable reference 16. Upon processing of the imaged echo data sets, a sectional mapping of the anatomical feature can be outputted, such as shown in FIG. 3B. In this specific embodiment, the open cuff body 17 is wrapped about a patient's thigh T and accordingly the mapping of FIG. 3B shows a representation of the femur F. Also shown in this figure is a representation the sciatic nerve SN, as an example of a clocking landmark. By monitoring the relative positioning of the representations of the femur F and of the sciatic nerve SN in the mapping of FIG. 3B, any rotation of the femur F or of the ultrasound tracking device 10 about the anatomical axis A can be monitored over time, which can provide an elegant way to navigate a femoral cut during surgery. It is noted that a significant portion of the ultrasound energy generated by some of the ultrasound probe units 14 may propagate directly through the patient's thigh to be measured by some diametrically opposite ultrasound probe units 14, or reflected back towards the emitting ultrasound probe units 14 by the femur F, for instance. However, it was found that at least a portion of the ultrasound energy may propagate sideways within the patient's thigh T and more specifically along the femur F which can act as an ultrasound waveguide. In these embodiments, at least some ultrasound signal portion 23 may be guided along a length of the femur F towards an extremity E thereof where the guided ultrasound signal portion 23 is at least partially reflected such that a reflected ultrasound signal portion 25 travels back towards the ultrasound tracking device 10.

In some embodiments, two axially spaced-apart ultrasound probe units 14 can be used to determine the ultrasound speed of either one of the guided ultrasound signal portions 23 and 25. In some embodiments, the ultrasound speed V can be determined based on the equation V=D1/T1, where D1 denotes an axial distance separating the two axially spaced-apart ultrasound probe units 14 and T1 denotes a first time duration elapsed between the detection of the guided ultrasound signal portion by a first one of the ultrasound probe units 14 and the detection of the guided ultrasound signal portion by the second one of the ultrasound probe units 14. In some other embodiments, the ultrasound speed V is not necessarily a measured ultrasound speed but rather a reference ultrasound speed retrieved from an accessible computer memory where it is stored. In either case, the ultrasound speed V of the guided ultrasound signal portion can be useful to determine the axial position of the ultrasound tracking device 10 with respect to the femur F. To do so, one may monitor a second time duration T2 elapsed between the generation of the ultrasound signal portion 23 within the patient's thigh T by a given ultrasound probe unit, and the detection of the reflected ultrasound signal portion 25 by the same ultrasound probe unit, or by another ultrasound probe unit sharing the same axial position along the femur F. By correlating the second time duration T2 to the ultrasound speed V discussed above, a propagation distance D2 can be determined. This propagation distance D2 can be indicative of the axial position of the ultrasound tracking device 10 along the patient's thigh T during the surgery. More specifically, the propagation distance D travelled by the ultrasound signal guided along the femur F may be given by D2=V×T2. In these embodiments, the axial position Pa of the ultrasound tracking device 10 with respect to the extremity E of the femur F would be half that propagation distance D2, i.e., Pa=D2/2. Accordingly, in these embodiments, the tracking of the ultrasound tracking device 10 using the trackable reference 19 may be omitted as the position of the ultrasound tracking device 10 can be otherwise tracked using ultrasound signals guided to and from the femur F, or any other bone under surgery. In some embodiments, additional computations and/or referencing may be required to distinguish which bone extremity reflects first. In these embodiments, determining which bone extremities reflects first or second can help to correctly position the bone or otherwise anatomical feature in the reference coordinate system.

Referring to FIGS. 4 to 7, different configurations using ultrasound tracking devices 10 are shown, in the context of knee surgery, and thus relative to a femur F and a tibia T, shown by their axes. The expressions femur F and tibia T are used as the CAS tracking system may track their axes. However, the ultrasound tracking devices 10 may actually be mounted onto the thigh and shank. For simplicity, the ultrasound tracking devices 10 are shown schematically without the ultrasound probe units 14 and with schematic details, but ultrasound probe units 14 are present in all ultrasound tracking devices 10 of FIGS. 4 to 7. On the other hand, the ultrasound tracking devices 10 of FIGS. 4 to 7 may be with or without reference markers 16, such that the visual presence or visual absence of the reference markers 16 is indicative of the presence or absence of the reference markers 16 on the ultrasound tracking devices 10.

Referring to FIG. 4, a first ultrasound tracking device 10A with reference marker 16 may be secured to the shank, and a second ultrasound tracking device 10B with reference marker 16 may be secured to the thigh. The readings from the ultrasound tracking devices 10 (i.e., ultrasound tracking devices 10A and 10B) may be used to perform a 2D reconstruction of the bones, and hence derive a location of the bone axis for each ultrasound tracking device 10. For one or both of the ultrasound tracking devices 10A and 10B, a mechanical member 30 may extend from the ultrasound tracking devices 10 to contact the limb at a position that is axially distanced from the wearable holder 12 of the ultrasound tracking devices 10, with “axially” referring to the respective axes F or T. The mechanical member 30 may be an arm that extends axially from the wearable holder 12 to provide a distal connection point 30A. As part of the ultrasound tracking devices 10, the mechanical member 30 may increase the axial footprint of the ultrasound tracking devices 10, and hence assist in blocking movement of the ultrasound tracking device 10 relative to the bone. In some embodiments, it can also be used for mechanical referencing. The mechanical member 30 may include a rigid component, such as an arm, and an attachment component, such as a strap, belt, etc. The attachment component of the mechanical member 30 may also be a pin or fastener connected to the bone. The connection made to the bone may involve an intermediate mechanical joint, such as a spherical joint, a universal joint, a hinge joint and the like. The intermediate mechanical joint can have a position sensor or encoder allowing the monitoring of the joint position prior, during and even after the surgery.

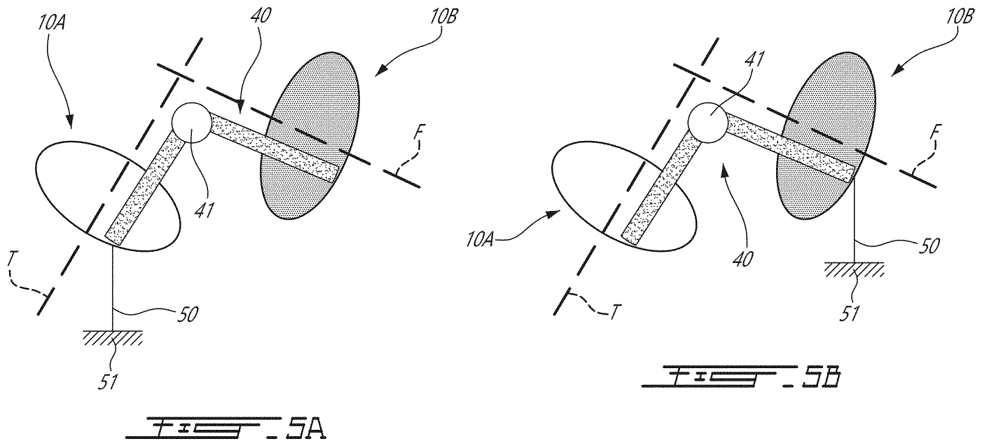

Referring to FIG. 5, a similar arrangement as in FIG. 4 is shown, with a first ultrasound tracking device 10A secured to the shank, and a second ultrasound tracking device 10B with reference marker 16 secured to the thigh. The first ultrasound tracking device 10A however does not have a reference marker 16. A linkage 40 interconnects the ultrasound tracking devices 10A and 10B. The linkage 40 may have a pair of rigid links 40A and 40B, respectively connected to the first ultrasound tracking device 10A and to the second ultrasound tracking device 10B. The links 40A and 40B are interconnected by a joint 41. The joint 41 may be a rotational joint constraining movement between the links 40A and 40B to a single degree of freedom (DOF). The joint 41 may be equipped with a sensor to determine an angular variation between the links 40A and 40B. For instance, the sensor is an encoder, but other possible embodiments include accelerometers and like inertial sensors. The mechanical members 30 may or may not be present in the configuration of FIG. 5.

In the embodiment of FIG. 5, the position and orientation of the first ultrasound tracking device 10A is calculated by the CAS tracking system using the position and orientation of the second ultrasound tracking device 10B, obtained using the reference marker 16, and using the geometry of the linkage 40 with the instant orientation between the links 40A and 40B as output by the sensor on the joint 41. Therefore, by the mechanical connection between ultrasound tracking devices 10A and 10B, the CAS tracking system may determine the position and orientation of the ultrasound tracking device 10A and consequently the location of axis T or other feature of the limb. The assembly of ultrasound tracking devices 10A and 10B as in FIG. 5 may be referred to as a knee brace, if the assembly is used at the knee. If the reference marker 16 is an optical marker, the assembly reduces the visual tracking envelope that must be covered by the position sensing system of FIG. 1, as a single optical marker must be tracked (or optical markers on a single ultrasound tracking device 10), though more optical markers could be used as well.

A reverse arrangement may be possible for FIG. 5, in which the first ultrasound tracking device 10A would be with a reference marker 16, and the second ultrasound tracking device 10B would be without.

Referring to FIGS. 5A and 5B, another embodiment is shown. The embodiment is similar to the arrangement of FIGS. 3 and 4, in that a first ultrasound tracking device 10A is secured to the shank, and a second ultrasound tracking device 10B is secured to the thigh. However, the ultrasound tracking devices 10A and 10B may be without reference markers 16. As in the embodiment of FIG. 5, the embodiments of FIGS. 5A and 5B feature the linkage 40 interconnecting the ultrasound tracking devices 10A and 10B. The linkage 40 has rigid links 40A and 40B, respectively connected to the first ultrasound tracking device 10A and to the second ultrasound tracking device 10B. The links 40A and 40B are interconnected by the joint 41, constraining movement between the links 40A and 40B to a rotational single degree of freedom (DOF). The joint 41 may be equipped with a sensor to determine an angular variation between the links 40A and 40B. For instance, the sensor is an encoder, but other possible embodiments include accelerometers and like inertial sensors. The mechanical members 30 may or may not be present in the configuration of FIGS. 5A and 5B. It is intended that the joint 41 may allow a single DOF in some embodiments. However, in some other embodiments, the joint 41 may be multi-directional, allowing movement in more at least two DOFs. In such embodiments, the sensor may be configured to monitor movement of the joint 41 in any of the corresponding DOFs.

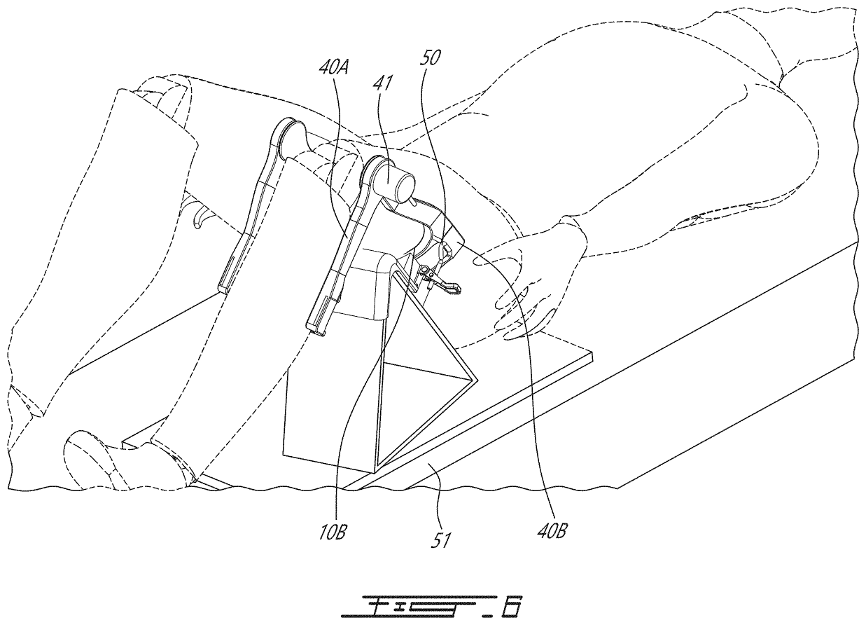

Still further in the embodiment of FIGS. 5A and 5B, a rigid connection 50 is present between one or both of the ultrasound tracking devices 10 or linkage 40, and a structure 51. In FIG. 5A, the rigid connection 50 is with the first ultrasound tracking device 10A. In FIG. 5B, the rigid connection is with the second ultrasound tracking device 10B. For instance, the structure 51 is a table, or a leg support, such as a leg support secured to a table, as in FIG. 6. The rigid connection 50 may include clamps, straps, etc., to secure the anatomical feature(s) to the structure 51, so as to inhibit or reduce movement of the anatomical feature(s) relative to the structure 51.

In the embodiment of FIG. 5A, the position and orientation of the first ultrasound tracking device 10A is calculated by the CAS tracking system using the position and orientation of the ultrasound tracking device 10A and structure 51, obtained for instance by 3D imaging of the limb as secured to the structure 51, to position the ultrasound tracking device 10A in the referential coordinate system of the structure 51, as permissible due to rigid connection 50 between the ultrasound tracking device 10A and the structure 51. Still for the embodiment of FIG. 5A, the position and orientation of the second ultrasound tracking device 10B is calculated by the CAS tracking system using the position and orientation of the first ultrasound tracking device 10A and the geometry of the linkage 40 with the instant orientation between the links 40A and 40B as output by the sensor on the joint 41. The rigid connection 50 of the embodiment of FIG. 5A may include a boot to which the shank is secured.

In the embodiment of FIG. 5B, the position and orientation of the second ultrasound tracking device 10B is calculated by the CAS tracking system using the position and orientation of the ultrasound tracking device 10B and structure 51, obtained for instance by 3D imaging (e.g., scan) of the limb as secured to the structure 51, to position the ultrasound tracking device 10B in the referential coordinate system of the structure 51, as permissible due to rigid connection 50 between the ultrasound tracking device 10B and the structure 51. Still for the embodiment of FIG. 5B, the position and orientation of the first ultrasound tracking device 10A is calculated by the CAS tracking system using the position and orientation of the second ultrasound tracking device 10B and the geometry of the linkage 40 with the instant orientation between the links 40A and 40B as output by the sensor on the joint 41.

For the embodiments of FIGS. 5A and 5B, by the mechanical connection between the ultrasound tracking devices 10A and 10B, the CAS tracking system may determine the position and orientation of the ultrasound tracking devices 10A and 10B and consequently the location of axes T and F or other features of the limbs.

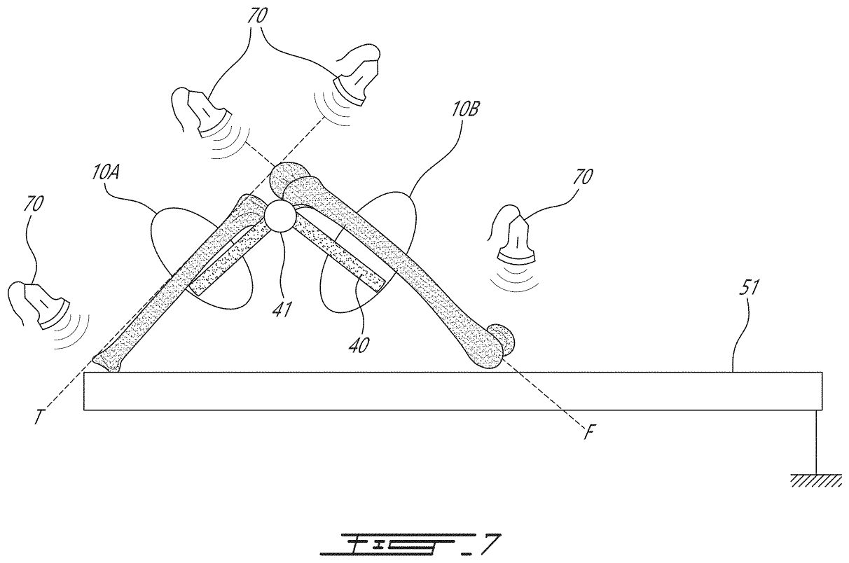

Referring to FIG. 7, another embodiment is shown. The embodiment is similar to the arrangement of FIG. 5, in that a first ultrasound tracking device 10A is secured to the shank, and a second ultrasound tracking device 10B is secured to the thigh, without reference markers 16. As in the embodiment of FIG. 5, the embodiment of FIG. 7 features the linkage 40 interconnecting the ultrasound tracking devices 10A and 10B. The linkage 40 has rigid links 40A and 40B, respectively connected to the first ultrasound tracking device 10A and to the second ultrasound tracking device 10B. The links 40A and 40B are interconnected by the joint 41, constraining movement between the links 40A and 40B to a rotational single degree of freedom (DOF). The joint 41 may be equipped with a sensor to determine an angular variation between the links 40A and 40B. For instance, the sensor is an encoder, but other possible embodiments include accelerometers and like inertial sensors. The mechanical members 30 may or may not be present in the configuration of FIG. 7.

In FIG. 7, ultrasound probe units 70 are distributed in the vicinity of the limbs, and the transducers in the ultrasound probe units 70 may be single-element or multi-element transducers, i.e., similar to the ultrasound probe units 14. Some or all of the ultrasound probe units 70 may be operated in a phase-array configuration. The ultrasound probe units 70 may be fixed in the referential system of the structure 51, for example. Although not shown as concealed, one or more ultrasound probe units 70 may be integrated into the table or bed. Stated differently, the position and orientation of the ultrasound probe units 70 is known in the coordinate system of CAS tracking system, such that readings from the ultrasound probe units 70 may be as a function of the coordinate system of the CAS tracking system.

In the embodiment of FIG. 7, the position and orientation of the first ultrasound tracking device 10A and of the second ultrasound tracking device 10B, is calculated by the CAS tracking system using the readings from the ultrasound probe units 70, and using optionally the geometry of the linkage 40 with the instant orientation between the links 40A and 40B as output by the sensor on the joint 41. Therefore, the CAS tracking system may determine the position and orientation of the axes T or F, other feature of the limb, without the optical tracking technology.

Although the position and orientation of the ultrasound probe units 70 are fixed in the embodiment described with reference to FIG. 7, it need not be the case. The position and orientation of the ultrasound probe unit(s) can be tracked in real time or quasi-real time in some other embodiments. As such, in another aspect of the present disclosure, there is provided an ultrasound tracking device for tracking a position and orientation of an anatomical feature during computer-assisted surgery. The anatomical feature can be one or more bone(s) such as the femur, the tibia, the fibula, spine/vertebrae, bone assembly(ies) such as the spine, organ(s), nerve(s) such as the sciatic nerve, artery(ies) such as the femoral artery, vein(s) such as the femoral vein, or any other suitable anatomical feature(s) of interest in computer-assisted surgery.

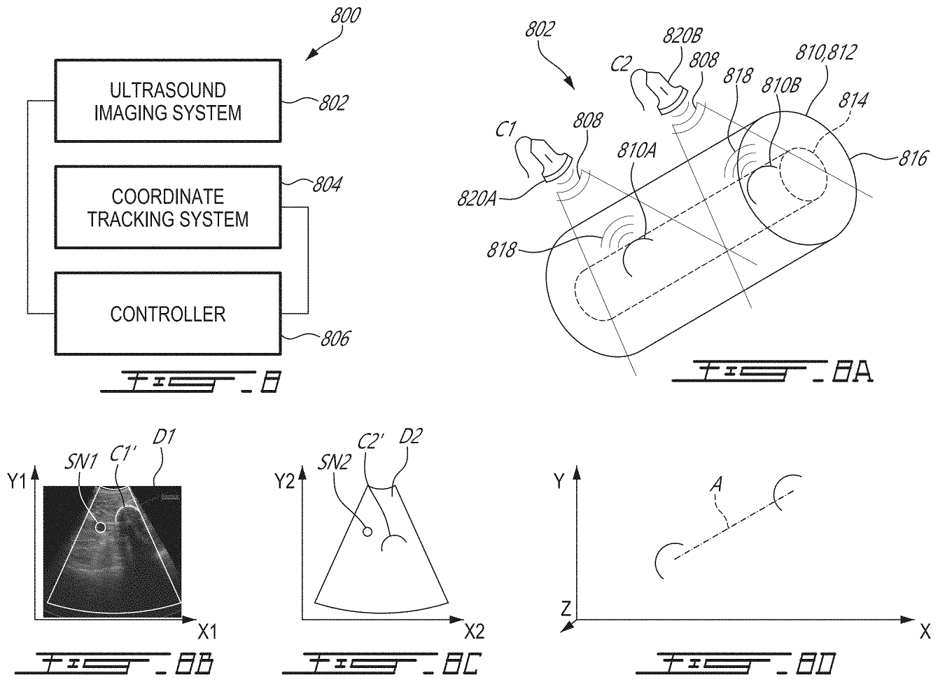

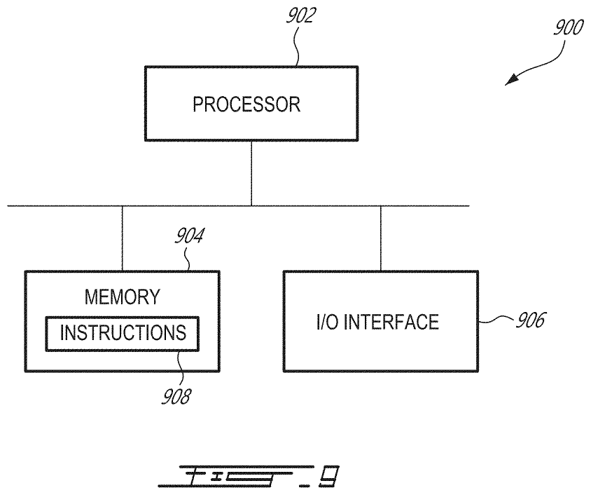

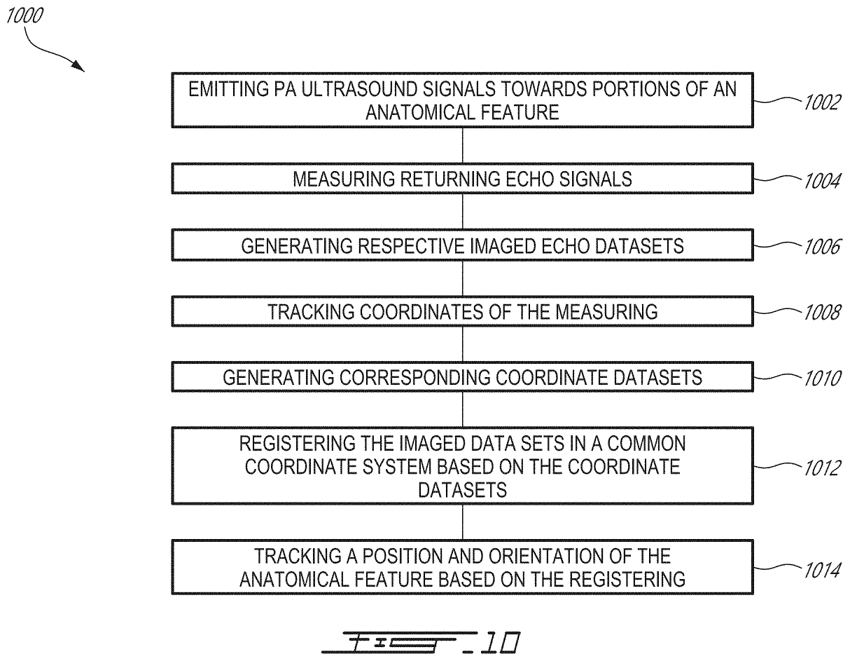

Referring now to FIG. 8, an example of an ultrasound tracking system is generally illustrated at 800. As depicted, the ultrasound tracking system 800 has an ultrasound imaging system 802 and a coordinate tracking system 804 which are communicatively coupled to a controller 806. The communication between these components can be wired, wireless or a combination of both depending on the embodiment. The ultrasound tracking system 800 can be part of a broader CAS tracking system such as the one described with reference to FIG. 1.

As best shown in FIG. 8A, the ultrasound imaging system 802 is adapted for emitting ultrasound signals 808 towards at least two portions 810a and 810b of an anatomical feature 810, in this case a limb 812 having an elongated bone 814 surrounded with bodily tissue 816. The two portions 810a and 810b that are interrogated by the ultrasound signals 808 in this example are two portions of the same elongated bone 814, but at different axial positions thereof, with the axial orientation following the length of the bone 814 in order to increase angular accuracy. However, in some other embodiments, the two portions can be associated with two different bones of a same bone assembly. For example, two or more vertebrae of a spine can be imaged by the ultrasound imaging system 802. Once emitted, the ultrasound signals 808 propagate towards the portions 810a and 810b until they are partially or wholly reflected by the internal structure of the anatomical feature 810 to form echo signals 818, as discussed above. The ultrasound imaging system 802 is adapted for receiving and measuring the echo signals 818 returning from the portions 810a and 810b of the anatomical feature 810, and for generating respective imaged echo datasets D1 and D2 (FIGS. 8B and 8C, respectively).

In the illustrated embodiment, the ultrasound imaging system 802 has at least two spaced-apart ultrasound probe units 820A and 820B each proximate a respective one of the portions 810a and 810b of the anatomical feature 810 of interest. The ultrasound probe units 820A and 820B are shown spaced from the limb, but can be in contact with the soft tissue, such as in the embodiments of FIGS. 2 to 7. Each of the ultrasound probe units 820A and 820B can generate its corresponding imaged echo dataset D. For instance, the first ultrasound probe unit 820A may have coordinates C1 as it is operated to generate the first imaged echo dataset D1, and the second ultrasound probe unit 820B may have coordinates C2 as it is operated to generate the second imaged echo dataset D2. As such, the ultrasound probe units 820A and 820B may be independent from one another. The ultrasound probe units 820A and 820B may be operated in a simultaneous manner, where the emission of the ultrasound signals 808, the measurement of the echo signals 818, and the generation of the corresponding imaged echo datasets D are synchronized, or in a sequential manner, where the ultrasound probe units 820A and 820B may be operated at different moments in time. Although two ultrasound probe units 820A and 820B are shown in this embodiment, more than two ultrasound probe units can be used in some other circumstances. In some other embodiments, the ultrasound imaging system 802 may have a single ultrasound probe unit 820A or 820B which is movable between the two portions 810A and 810B of the anatomical feature 810 to probe them in a sequential manner. In these latter embodiments, the single ultrasound probe unit may be used to generate the corresponding imaged echo datasets D1 and D2 sequentially, i.e., at different moments in time t1 and t2.

The imaged echo datasets D1 and D2 can be expressed in any suitable format. Examples of such imaged echo datasets D1 and D2 are plotted at FIGS. 8B and 8C for understanding purposes. As shown, the first imaged echo dataset D1 is plotted in a first coordinate system X1, Y1 in FIG. 8B, whereas the second imaged echo dataset D2 is plotted in a second coordinate system X2, Y2 in FIG. 8C, where representations of the imaged portions of the imaged bone can be seen. As shown, the imaged portions each have corresponding coordinates C1′ and C2′ in their respective coordinate systems. It is noticed that in both imaged echo data datasets shown a bone portion but also a sciatic nerve portion. Accordingly, in this embodiment, the anatomical feature can be tracked by the tracking of the bone and/or by the tracking of the sciatic nerve or the known relative anatomical feature can provide greater angular accuracy since it is further from bone and a small rotation around the bone may be more easily detected.

Referring back to FIG. 8, the coordinate tracking system 804 is adapted for tracking coordinates of the ultrasound imaging system 802, and more specifically ultrasound probe unit(s) thereof, during the measurement of the echo signals, and for generating corresponding coordinate datasets. The coordinate datasets can include coordinates pertaining to a position and orientation of the ultrasound probe unit(s) as they are operated, coordinates pertaining to a plane along which the ultrasound signals are emitted and along which the echo signals are received, or both depending on the embodiment. In some embodiments, the coordinate tracking system 804 is an optical coordinate tracking system which optically tracks a position and orientation of the ultrasound imaging system 802, via one or more reference markers fixedly attached to the ultrasound imaging system 802 using one or more cameras, or via image processing of images from cameras, such as 3D depth cameras. Alternatively or additionally, the coordinate tracking system can be a mechanical coordinate tracking system which tracks the position and orientation of the ultrasound imaging system 802 relative to a frame using sensors of the encoder type, for instance. In some embodiments, the coordinate tracking system 804 can involve self-locating sensors, such as one or more GPS sensors, one or more accelerometers, one or more gyroscopes, or other inertial sensors, and the like. In some embodiments, the self-locating sensors do not necessarily have a fixed relationship with one another but can nonetheless find each other.

Examples of some coordinate tracking systems 804 are described below.

It is noted that, in view of the above, coordinates of the ultrasound imaging system 802 are tracked during its operation. Accordingly, the controller 806 is configured for registering the imaged echo datasets D to one another in a common coordinate system X, Y, Z based on the coordinate datasets generated by the coordinate tracking system 804. Such registered imaged echo datasets are schematically illustrated in FIG. 8D for exemplary purposes only. As shown, determining an axis A of the tracked anatomical feature may be facilitated by having the imaged echo datasets registered relative to one another in the common coordinate system X, Y, Z. In some embodiments, the common coordinate system X, Y, Z can correspond to a reference coordinate system of a CAS tracking system such as the one described above with reference to FIG. 1. Still referring to FIG. 8, the controller 806 can thereby track the position and orientation of the anatomical feature from the imaging performed by the ultrasound probe unit(s) and from the tracking of ultrasound probe unit(s) during that imaging, if applicable. In some embodiments, the ultrasound imaging system 802 can be operated to probe a significant number of portions of the anatomical feature, thereby allowing a construction of a model of the anatomical feature, which may be of interest in computer-assisted surgery. The measurements can constantly be transferred to the controller 806 for the position and orientation of the anatomical feature in space to be updated in real time or quasi-real time.

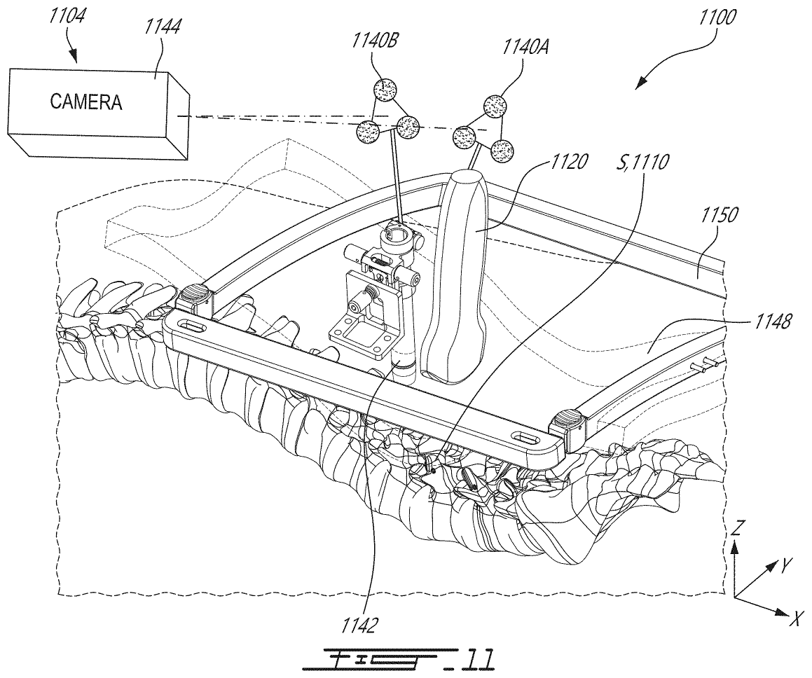

The controller 806 can be provided as a combination of hardware and software components. The hardware components can be implemented in the form of a computing device 900, an example of which is described with reference to FIG. 9. Moreover, instructions to be executed by the controller 806 can be implemented in the form of one or more software applications.

Referring to FIG. 9, the computing device 900 can have a processor 902, a memory 904, and I/O interface 906. Instructions 908 for tracking a position and an orientation of an anatomical feature during computer-assisted surgery can be stored on the memory 904 and accessible by the processor 902.

The processor 902 can be, for example, a general-purpose microprocessor or microcontroller, a digital signal processing (DSP) processor, an integrated circuit, a field-programmable gate array (FPGA), a reconfigurable processor, a programmable read-only memory (PROM), a system on a chip, an embedded controller, or any combination thereof.

The memory 904 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 906 enables the computing device 900 to interconnect with one or more input devices, such as the ultrasound imaging system or its ultrasound probe unit(s), the coordinate tracking system, keyboard(s), mouse(s), or with one or more output devices such as display screen(s), computer memory(ies), network(s) and like.

Each I/O interface 906 enables the controller 806 to communicate with other components, to exchange data with other components, to access and connect to network resources, to server applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX, Zigbee), SS7 signaling network, fixed line, local area network, wide area network, and others, including any combination of these.

It is intended that the instructions 908 may be executed to receive the imaged echo dataset(s), receive the coordinate dataset(s), to register the received imaged echo datasets in the common coordinate system X, Y, Z and to track the position and orientation of the anatomical feature. In some embodiments, a software application to execute the instructions 908 is stored on the memory 904 and accessible by the processor 902 of the computing device 900. In some embodiments, the software application can be locally or remotely stored.

The computing device 900 and the instructions to be executed described above are meant to be examples only. Other suitable embodiments of the controller 806 can also be provided, as it will be apparent to the skilled reader.

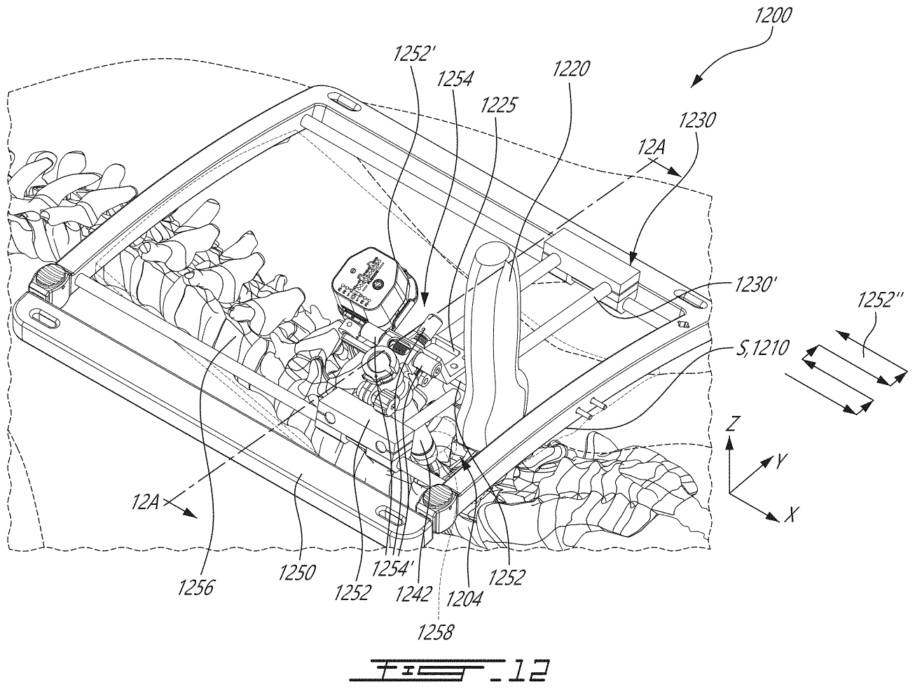

FIG. 10 shows a flow chart of an example method 1000 for tracking a position and orientation of an anatomical feature in computer-assisted surgery. As some of the steps of the method 1000 can be performed by the ultrasound imaging system or by the coordinate tracking system, some other steps of the method 1000 can be performed by the controller.

At step 1002, ultrasound signals are successively emitted towards different portions of the anatomical feature of interest. The ultrasound signals may be emitted by a single phased-array ultrasound probe unit in some embodiments, while they may be emitted by different phased-array ultrasound probe units in some other embodiments. The emission of the ultrasound signal may therefore be simultaneous or sequential depending on the embodiment.

At step 1004, echo signals returning from the portions of the anatomical feature are received and measured. The returning echo signals may be measured by a single ultrasound probe unit in some embodiments, while they may be measured by different ultrasound probe units in some other embodiments. Accordingly, the measurement of the returning echo signals may therefore be simultaneous or sequential depending on the embodiment.

At step 1006, imaged echo datasets associated to each measured echo signals are generated. In a variant, the imaged echo datasets are generated by one or more ultrasound imaging device(s) of the ultrasound imaging system which is communicatively coupled to the ultrasound probe unit(s). As such, the signal measured by the ultrasound probe unit(s) may be converted into the imaged echo datasets by the ultrasound imaging device(s) upon reception or subsequently, depending on the embodiment. The imaged echo datasets may be stored on a non-transitory computer-readable memory so as to be accessible by a processor of the controller at a subsequent step of the method 1000. Each ultrasound probe unit may have its corresponding ultrasound imaging device in some embodiments. In some other embodiments, a single ultrasound imaging device may be communicatively coupled to the ultrasound probe units. The imaged echo datasets may have corresponding probe unit stamps identifying their source ultrasound probe unit. The imaged echo datasets may have corresponding time stamps identifying at what moment in time the echo signals they represent have been measured.

As discussed above, the steps 1002, 1004 and 1006 may be performed simultaneously or sequentially, depending on whether an ultrasound imaging system having one or more ultrasound probe units or one or more ultrasound imaging devices is used.

At step 1008, optionally, coordinates of the ultrasound imaging system are tracked during at least the measurement step 1004. The coordinates of the ultrasound imaging system may be tracked only during any one of the steps 1002, 1004 and 1006 or during all of the steps 1002, 1004 and 1006 depending on the embodiment. In some embodiments, the coordinates of the ultrasound imaging system are tracked in a continuous manner during a computer-assisted surgery, concurrently with the steps 1002 and/or 1004. Steps 1002, 1004, 1006 may occur in real-time or quasi-real-time. However, in some other embodiments, it may be preferred to track the ultrasound imaging system only at specific moments in time. In the latter embodiments, the ultrasound imaging system and the coordinate tracking device may be synchronized to one another. As such, the coordinate tracking device may be triggered shortly before, during, and/or after the operation of the ultrasound imaging system.

In step 1010, coordinate datasets indicative of the tracked coordinates of the ultrasound imaging system are generated. The coordinate datasets may have corresponding probe unit stamps identifying which one of the ultrasound probe units is tracked. The coordinate datasets may have corresponding time stamps identifying at what moment in time the ultrasound probe units have been tracked. As such, it can be possible to find a correspondence between the imaged echo datasets and the coordinate datasets. Each coordinate dataset may include information relating to the geometric relationship between the portion of the ultrasound probe unit that is being tracked and the plane along which the ultrasound imaging is performed. For instance, in embodiments where a reference marker is mounted on a top of an ultrasound probe unit, the geometric relationship can be based on the length of the ultrasound probe unit, the length at which the reference marker extends from the top of the ultrasound probe unit, the shape of the ultrasound probe unit, and the like.

At step 1012, the imaged echo datasets are registered to one another in a common coordinate system based on the coordinate datasets. In some embodiments, the registering may involve the use of the probe unit stamps and/or the time stamps so as to ensure that the corresponding imaged echo datasets be registered in the common coordinate system using the corresponding coordinate dataset. If the anatomical features are fixed in the coordinate system, and the ultrasound units are also fixed, the steps 1008 and 1010 may be done punctually, at intervals, etc.

At step 1014, the position and orientation of the anatomical feature are tracked based on the registering step 1012. The tracking of the anatomical feature can include a step of calculating an axis of the tracked anatomical feature based on its position and orientation.

In some embodiments, the step 1012 of registering includes a step of generating an anatomical feature model representative of the anatomical feature based at least on the imaged echo datasets and corresponding coordinate datasets. The anatomical feature model can therefore be generated on the go in the coordinate system X, Y and Z. As such, the anatomical feature model can be incrementally improved as more imaged echo datasets and corresponding coordinate datasets are received over time.

In some embodiments, the step of generating the anatomical feature model can include a step of accessing a reference model base. For instance, a reference model base may be selected in a database comprising different reference model bases such as a tibia model base, a femur model base, a spine model base including, e.g., a sacral, lumbar, thoracic or cervical model base(s), a shoulder joint model base, a humerus model base, a scapula model base, a forearm model base, a pelvis model base, an elbow joint model base and the like. In embodiments where the femur is under surgery, for instance, a reference model base associated to the femur may be selected. As such, the femur reference model base may be positioned and orientated in the coordinate system based on the imaged echo datasets and corresponding coordinate datasets. In some other embodiments, the step of accessing a model base can include a step of fetching a patient-specific model which can be based on pre-operative or peri-operatively images of the anatomical feature of a given patient obtained using various imaging modalities. For example, the patient-specific anatomical models may have been generated from magnetic resonance imagery, or from radiography in its various forms, as possibilities. In these embodiments, the patient-specific model may be positioned and oriented in the coordinate system based on the imaged echo datasets. Generic models may be used as well, such as those from a bone atlas.

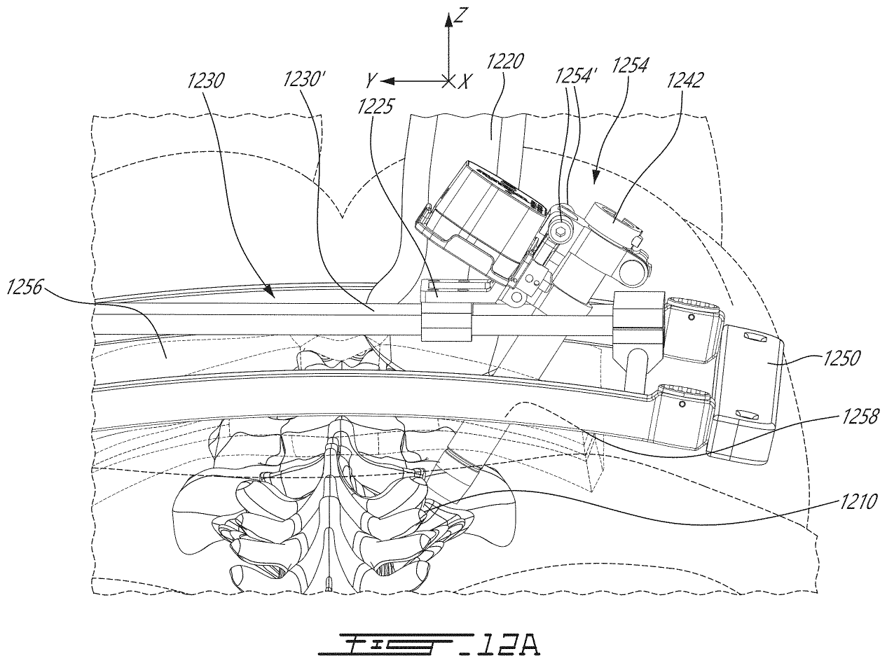

Referring now to FIG. 11, a portion of an ultrasound tracking system 1100 used in the context of a spine surgery is shown. As depicted, a single ultrasound probe unit 1120 is provided along with an optical coordinate tracking system 1104. The optical coordinate tracking system 1104 has a first reference tracker 1140A mounted to the ultrasound probe unit 1120 and a second reference tracker 11406 mounted to a surgical tool holder 1142. The illustrated tracking modality is one among others that can be used (3D camera being another), with a rigid connection of the ultrasound probe unit 1120 and of the surgical tool holder 1142 to the frame 1150 being an option as well. The optical coordinate tracking system 1104 may also have a camera 1144 imaging the first and second reference trackers 1140A and 1140B during the computer-assisted surgery. The tracking system may also be inertial.

In some embodiments, the ultrasound probe unit 1120 may be moved in any given pattern to map desired portions of the anatomical feature 1110 which is a spine in this specific embodiment. In some embodiments, the pattern may be arbitrary as long as all the desired portions of the anatomical feature 1110 have satisfactorily been probed.

In this specific embodiment, the imaged echo datasets can be registered to one another in the common coordinate system so as to construct an anatomical feature model in a gradual manner as the arbitrary scan is being performed by an operator, for instance. In some other embodiments, a generic or patient-specific spine base model may be retrieved to build thereon to increase resolution.

As can be expected, the position and orientation of the surgical tool holder 1142 are also tracked in this embodiment. Accordingly, once the position and orientation of the anatomical feature 1110 are suitably tracked by the ultrasound tracking system, the surgical tool holder 1142 holding a surgical tool may be moved as desired in the common coordinate system to perform at least some surgical steps, in some embodiments.

An acoustically transmissive material such as ultrasound gel 1148 may be applied onto the outer-skin surface S of the anatomical feature 1110. In these embodiments, the transmission of the ultrasound signal from the ultrasound probe unit 1120 to the outer-skin surface S, or of the echo signal from the outer-skin surface S back towards the ultrasound probe unit 1120, may be enhanced.

In some embodiments, the ultrasound probe unit 1120 and the surgical tool holder 1142 can be mounted to a frame 1150 in accordance to a known geometric relationship, which can ease the registering of the imaged echo datasets and the coordinates datasets in the common coordinate system X, Y, Z. If the patient and the frame 1150 are fixed in the coordinate system, the ultrasound probe unit 1120 and the surgical tool holder 1142 may not need to be tracked optically. In some embodiments, intraoperative imaging may be eliminated from the procedure as the ultrasound tracking system 1100 may be used to compute (or recompute) an X-ray like image from the imaged data sets generated by the ultrasound probe unit 1120 in real time or quasi real time.

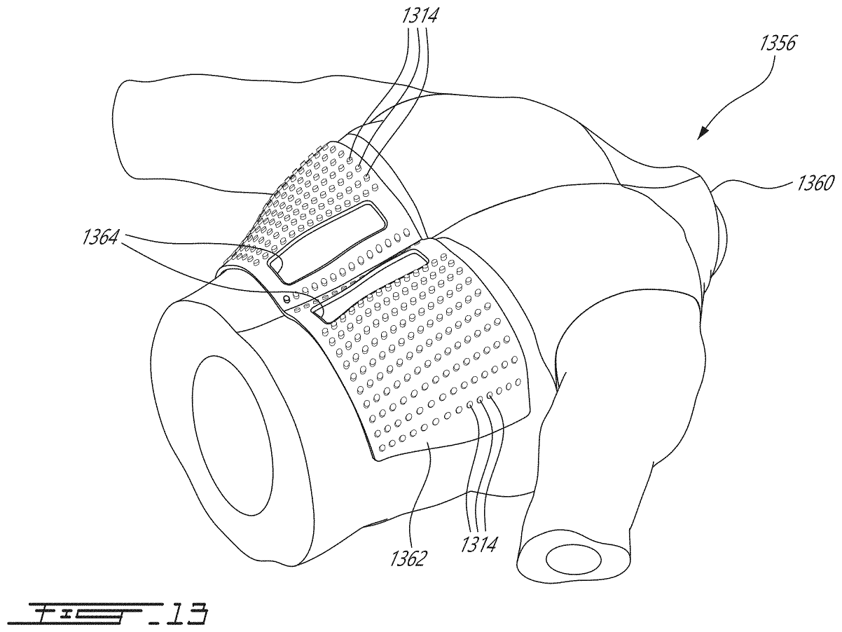

Referring now to FIGS. 12 and 12A, a portion of another example ultrasound tracking system used in the context of a spine surgery is shown at 1200. As depicted, the ultrasound tracking system 1200 has an ultrasound probe assembly including a single ultrasound probe unit 1220, and a mechanical coordinate tracking system 1204. The ultrasound probe unit 1220 may be mounted to the mechanical coordinate tracking system 1204 by a bracket 1225, so as to be rotatable relative to a bar 1230′ of a carriage 1230 of the mechanical coordinate tracking system 1204. In an embodiment, a cylindrical joint is present between the bracket 1225 and the bar 1230′, such that the ultrasound probe unit 1220 may rotate about axis Y, and slide in a direction parallel to axis Y, relative to the bar 1230′. In an embodiment, the bracket 1225 is only pivotable. In some embodiments, the bar and carriage assembly can be motorized. The bracket 1225 and the carriage 1230 can be motorized as well in some other embodiments.

The mechanical coordinate tracking system 1204 has a frame 1250 to which the ultrasound probe unit 1220 is movably mounted via the carriage 1230 and which is fixedly mounted relative to the anatomical feature 1210. For instance, the frame 1250 may be directly fixed to the patient in some embodiments. The frame 1250 may be fixedly mounted to a table on which the patient lies in some other embodiments. The frame 1250 may be mounted to any other suitable structure as circumstances may dictate. The mechanical coordinate tracking system 1204 also has position sensors 1252 associated with the carriage 1230 and/or the frame 1250, to measure the movement of the ultrasound probe unit 1220 with respect to the frame 1250, especially the X-Y position. The position sensors 1252 may be range finders, encoders, linear actuators, etc, such that the X,Y coordinates of the ultrasound probe unit 1220 and the surgical tool and holder 1242 may be known relative to the frame 1250, and thus in the coordinate system. As observed, an inertial sensor unit 1252′ (e.g., iASSIST® sensor) may be present to determine the orientation of the surgical tool and holder 1242. In the illustrated embodiment, the surgical tool and holder 1242 is also mounted to the bracket 1225, so as to move concurrently with the ultrasound probe unit 1220, with the tool in the holder 1242 being in the field of imaging of the ultrasound probe unit 1220. For example, the surgical tool and holder 1242 may include a drill guide that can be tracked in position via the frame 1250 and position sensors 1252, and in orientation (phi, theta, ro) via the inertial sensor unit 1252′. Rotary encoders or like sensors can be an alternative to the inertial sensor unit 1252′. As observed, the position and orientation of the surgical tool and holder 1242 may be adjusted relative to the bracket 1225, by way of adjustable support 1254. For instance, a drill guide of the surgical tool and holder 1242 may be moved axially, and its orientation can be adjusted via dials 1254′, as an option. In some embodiments, the rotation about the Y-axis happens between the adjustable support 1254 by way of a rotating mechanism (e.g., a rack and pinion assembly) controlled by the pair of aligned dials 1254′. By doing so, the holder 1242 may be tilted as desired. The rotation about the x-axis can happen between the bracket 1225 and the adjustable support 1254 through the single knob 1254′.

In some embodiments, the ultrasound probe unit 1220 may be moved in any given pattern to map desired portions of the anatomical feature 1210. In some embodiments, the pattern may be a raster scan pattern 1252″ in which the ultrasound probe unit 1220 is moved along a first direction in the X-axis via movement of the carriage 1230 relative to rails of the frame 1250, then incrementally along the Y-axis via the bracket 1225 and its cylindrical joint, then along a second direction opposite the first direction in the X-axis, and so forth, until all the desired portions of the anatomical feature 1210 have been satisfactorily probed. As can be expected, the position sensors 1252, which may be of the encoder type, can measure the movement of the ultrasound probe unit 1220 with respect to the frame 1250. As such, the echo signal datasets generated by the ultrasound probe unit 1220 can be registered to one another in the common coordinate system X, Y, Z based on the coordinate datasets measured by the position sensors 1252. The orientation of the drill guide of the surgical tool and holder 1242, or of any other tool, can be tracked in orientation, such as by the inertial sensor unit 1252′.

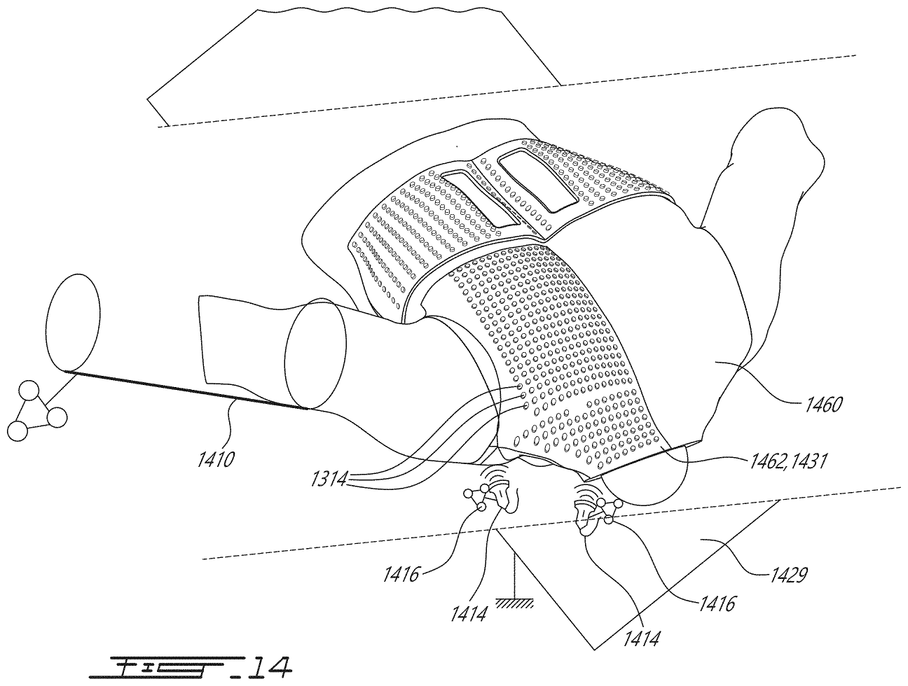

In some embodiments, the acoustically transmissive material may not be provided in the form of gel. Indeed, the acoustically transmissive material can be provided in the form of one or more solid pieces of material. In some embodiments, the acoustically transmissive material may be provided in the form of a wearable element 1356, such as a vest or corset, to be worn by the patient during the computer-assisted surgery. As depicted in this embodiment, the wearable element 1356 has one or more surgery openings 1364 allowing access for the surgical tool and holder 1342 to interact with the anatomical feature 1310, or its surroundings, without risks of acoustically transmissive gel reaching the inner body during the computer-assisted surgery. An example of such a wearable element 1356 is shown in FIG. 13. As shown, in this example, the wearable element 1356 has a garment, in this case a compression shirt 1360, and an ultrasound imaging interface 1362 being removably or fixedly attached to an exterior surface of the garment. In some embodiments, the garment 1360 tightly surrounds a portion of the body of the patient. In this way, the garment 1360 can be put on in a manner which suitable positions the ultrasound imaging interface 1362 at a satisfactory position of the body, in direct contact against the skin. The ultrasound imaging interface 1362 is for example made of a solid, semi-rigid or flexible acoustically transmissive material thereby enhancing ultrasound imaging capabilities, though the solid is flexible for the interface 1362 to conform to the surface of the skin. As shown, the wearable element 1356 has two parallel elongated surgery openings 1364 which are located above a lumbar region of the vest and allowing access to lumbar vertebrae in this embodiment, in the standard orientations for tools accessing the vertebrae. In some embodiments, the tools including, but not limited to, drills, awls and the like, may be used as ultrasound waveguides to transfer the ultrasound signal going to and incoming from a closest point of the anatomical feature. By using the tools as ultrasound waveguides, a more precise mapping of the anatomical feature may be achieved as the tools can be brought in closer to the bone wall, for instance, than the ultrasound probe units. The ultrasound tracking system 1200 of FIG. 12 may be used with the wearable element 1356, for example, with the ultrasound probe unit 1220 being applied against the central strip between the openings 1364. It is intended that the vest covers not only a back portion of the patient but also the lateral portion of the patient. In some embodiments, the lateral portion of the vest, corset or other bodily garment can be used as a support for implanting cages. Such a wearable element can be used in surgeries where access via the sides of the patient is required. More specifically, the ultrasound imaging interface 1362 can extend on the sides of the patient and may reach the coronal plane of the patient. In some embodiments, the ultrasound imaging interface 1362 can extend towards a lower back portion of the patient. In some other embodiments, the wearable element can be provided in the form of a tight-fitting sleeve, a belt, a ring, a strap or to any suitable element which can be worn by a limb, an appendage, or other anatomical features of the patient having a bone or other anatomical feature to be tracked.

In some embodiments, for instance with reference to FIGS. 13 and 14, the ultrasound probe units 1314 may be integrated at one or more positions of the ultrasound imaging interface 1362 to provide a wide coverage including frontal and sagittal plane scanning directions. It is intended that, as the ultrasound probe units 1314 are embedded at respective positions within the ultrasound imaging interface 1362, the amount of obstruction in the surgical field can be significantly reduced, thereby leaving more room for tool(s) to be manipulated proximate the surgery openings 1364 during the surgery. In some embodiments, the ultrasound probe units 1314 can be uniformly distributed in the ultrasound imaging interface 1362, with the probe unit density (i.e., number of probe units per area unit) varying from one embodiment to another. In some other embodiments, the embedded ultrasound probe units 1314 are installed along a semi-circular contour extending around either or both of the surgery openings 1364. In any case, the embedded ultrasound probe units 1314 can allow for the mapping of the anatomical feature as well as real-time monitoring of instruments and tools. In some other embodiments, the embedded ultrasound probe units 1314 can be omitted. In these embodiments, the ultrasound imaging may be performed using one or more handheld probe units being either fixedly or movably mounted to a carriage or free to be moved by hand by a skilled technician.

In some embodiments, wearable element(s) can be provided to help shoulder or hip surgeries as well. An example of such a wearable element 1456 used in shoulder surgeries is shown in FIG. 14. As shown, the wearable element 1456 has a garment 1460 to be worn by the patient, and an ultrasound imaging interface 1462 covering at least a portion of the garment 1460. As shown, the ultrasound imaging interface 1462 is made of a solid acoustically transmissive material and may have one or more surgery openings defined therein (not shown) allowing access to the bones of the shoulder. In this specific embodiment, the ultrasound imaging interface 1462 is provided in the form of a flexible mat 1431 covering the shoulder from the shoulder blade to the anterior portion of the pectoral muscles. As shown, in this embodiment, ultrasound probe units 1414 may be embedded in the flexible mat 1431.

In this embodiment, trackable references 1416 fixed to a reference bed can help track the positioning of the patient relative to reference bed 1429. In some embodiments, trackable references 1416 are positioned on movable ultrasound probe units 1414 to track the positioning of the shoulder with respect to the reference bed 1429 as well. In some embodiments, the ultrasound probe units 1414 are collectively used to monitor a rotation of the humerus about its anatomical axis by monitoring another feature of the patient's arm such as a vein or an artery, using one or more other ultrasound tracking devices 1410, for instance. Locating the scapula to orient/link the glenoid in the surgical plane may also be envisaged in some embodiments. Although the reference bed 1429 is featured under the patient's belly, it can also be used with the patient lying on his back on the reference bed 1429 for some other types of surgeries.

### EXAMPLE 1

In another aspect of the disclosure, there is described a wearable element for use in computer-assisted surgery involving ultrasound tracking of an anatomical feature of a patient, the wearable element comprising: a garment to be worn by the patient; and an ultrasound imaging interface covering at least a portion of the garment, the ultrasound imaging interface being made of a solid acoustically transmissive material and having one or more surgery openings defined therein allowing access to the anatomical feature.

In some embodiments, the garment is an upper-body garment such as a shirt, a vest, a corset, a sleeve, a belt and the like. As discussed above, ultrasound probe units may be embedded in the ultrasound imaging interface. In some embodiments, the garment can include adhesive pad(s) such as electrocauterisation grounding pads. In some embodiments, the ultrasound imaging interface covers at least a portion of a back portion of the upper-body garment. In some embodiments, the ultrasound imaging interface extends towards each lateral side of the upper-body garment and reaches at least a coronal plane of the upper-body garment. In some embodiments, the ultrasound imaging interface extends towards a lumbar portion of the upper-body garment and reaches at least a transverse plane of the upper-body garment. In some embodiments, the surgery opening(s) extend(s) along a spine orientation of the upper-body garment. In some embodiments, the ultrasound imaging interface has an ultrasound imaging strip following a spine orientation of the upper-body garment, and at least a surgery opening extends alongside and parallel to the ultrasound imaging strip. The ultrasound imaging strip allowing the imaging of the spine of the patient with an ultrasound probe unit being perpendicular to the ultrasound imaging strip, the surgery opening(s) extending alongside the ultrasound imaging strip allowing surgical tool(s) to reach the spine at an oblique angle through them. In some embodiments, the garment is made of a compression material tightly fitting the patient.

### EXAMPLE 2

In another aspect of the disclosure, there is described an ultrasound tracking device for use with a position sensing system to register position and orientation in computer-assisted surgery, the ultrasound tracking device comprising: a wearable holder adapted to be secured to an anatomic feature; at least two ultrasonic probe units supported by the wearable holder and adapted to emit signals to image part of the anatomic feature; at least one reference tracker supported by the wearable holder; and a mechanical member projecting from a remainder of the ultrasound tracking device and increasing an axial footprint of the ultrasound tracking device.

### EXAMPLE 3

In another aspect of the disclosure, there is described a set of ultrasound tracking devices for use with a position sensing system to register position and orientation in computer-assisted surgery, each of the ultrasound tracking device comprising: a wearable holder adapted to be secured to an anatomic feature, and at least two ultrasonic probe units supported by the wearable holder and adapted to emit signals to image part of the anatomic feature; at least one reference tracker supported by one of the wearable holders; and a linkage between the set of ultrasound tracking devices, the linkage having a rotational joint and a sensor for determining an angular value variation in the rotational joint.

### EXAMPLE 4

In another aspect of the disclosure, there is described an ultrasound tracking system for tracking a position of the ultrasound tracking device with respect to an extremity of an anatomical feature in computer-assisted surgery, the ultrasound tracking system comprising: at least an ultrasound probe unit fixedly mounted relative to the anatomical feature, the ultrasound probe unit being adapted for emitting an ultrasound signal within said anatomical feature, at least a portion of the ultrasound signal being guided away from the ultrasound probe unit and along an anatomical axis of the anatomical feature towards and the extremity thereof, the ultrasound probe unit detecting at least a reflected portion of the ultrasound signal being guided from the extremity of the anatomical feature and back towards the ultrasound probe unit; a controller being communicatively coupled to said ultrasound probe unit, said controller having a processor and a memory having stored thereon instructions that when executed by said processor perform the steps of: determining an axial position of the ultrasound probe unit relative to the extremity of the anatomical feature based on an ultrasound speed value indicative of a speed at which the portion of the ultrasound signal travels along the anatomical feature and on a time duration indicative of a time duration elapsed between the emitting and the detecting. In some embodiments, the ultrasound speed value is measured in situ based on measurements performed by at least two ultrasound probe units axially spaced-apart from one another along the anatomical axis.

While illustrated in the block diagrams as groups of discrete components communicating with each other via distinct data signal connections, it will be understood by those skilled in the art that the preferred embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present preferred embodiment. The embodiments of the invention described above are intended to be exemplary only. For instance, as knee or lumbar surgeries are described above, they are meant to be exemplary only. The methods and systems described herein can also be applicable in pelvis surgery, shoulder blade surgery, and any other bone or articulation surgery. Moreover, in some embodiments, the ultrasound methods and systems can be used to find tools, screws and other surgery equipment within the body of the patient during surgery. The scope of the invention is therefore intended to be limited solely by the scope of the appended claims.

1. An ultrasound tracking system for tracking a position and orientation of an anatomical feature in computer-assisted surgery, the ultrasound tracking system comprising:
an ultrasound imaging system having a phased-array ultrasound probe unit being adapted for emitting ultrasound signals successively towards different portions of said anatomical feature, measuring echo signals returning from said portions of said anatomical feature and generating respective imaged echo datasets;
a coordinate tracking system tracking coordinates of said ultrasound phased array probe unit during said measuring, and generating corresponding coordinate datasets; and
a controller being communicatively coupled to said ultrasound imaging system and said coordinate tracking system, said controller having a processor and a memory having stored thereon instructions that when executed by said processor perform the steps of:
registering said imaged echo datasets in a common coordinate system based on said coordinate datasets; and
tracking said position and orientation of said anatomical feature based on said registering.

2. The ultrasound tracking system of claim 1 wherein said registering includes generating an anatomical feature model of said anatomical feature based at least on said imaged echo datasets, and registering said anatomical feature model in said coordinate system based on said coordinate datasets.

3. The ultrasound tracking system of claim 2 wherein said generating said anatomical feature model includes accessing a reference model base, said generating said anatomical feature model being further based on said reference model base.

4. The ultrasound tracking system of claim 3 wherein said reference model base is selected from a group consisting of: a tibia model base, a femur model base, a spine model base, a shoulder joint model base, a humerus model base, a scapula model base, a forearm model base, a pelvis model base, and an elbow joint model base.

5. The ultrasound tracking system of claim 1 wherein said ultrasound imaging system has at least two spaced-apart phased-array ultrasound probe units each proximate a respective part of said anatomical feature and generating said imaged echo datasets, said coordinate tracking system tracking coordinates of each one of said at least two spaced-apart phased-array ultrasound probe units during said measuring.

6. The ultrasound tracking system of claim 1 further comprising displaying said tracked anatomical feature in real-time during the computer-assisted surgery.

7. The ultrasound tracking system of claim 1 wherein said ultrasound signals are successively steered towards said different portions of said anatomical feature.

8. The ultrasound tracking system of claim 1 wherein said ultrasound signals are successively focused towards said different portions of said anatomical features.

9. The ultrasound tracking system of claim 1 wherein said coordinate tracking system is an optical coordinate tracking system having a reference tracker mounted to said ultrasound imaging system, and a camera imaging at least said reference tracker during said measuring, said optical coordinate tracking system optically tracking said coordinates of said ultrasound imaging system based on said camera imaging.

10. The ultrasound tracking system of claim 1 wherein said coordinate tracking system is a mechanical coordinate tracking system having a frame to which said ultrasound imaging system is movably mounted, and at least a sensor sensing a relative movement between said ultrasound imaging system and said frame, said mechanical coordinate tracking system tracking said coordinates of said ultrasound imaging system based on said sensed relative movement.

11. The ultrasound tracking system of claim 1 further comprising a wearable element having an ultrasound imaging interface made of a solid acoustically transmissive material through which said ultrasound signals are propagated.

12. The ultrasound tracking system of claim 11 wherein said ultrasound imaging interface includes a surgery opening allowing access to said anatomical feature during said computer-assisted surgery.

13. A method for tracking a position and orientation of an anatomical feature in computer-assisted surgery, the method comprising:
emitting phased-array ultrasound signals towards different portions of said anatomical feature, measuring echo signals returning from said portions of said anatomical feature and generating respective imaged echo datasets while tracking coordinates of said ultrasound imaging system, and generating corresponding coordinate datasets; and
a controller performing the steps of:
registering said imaged echo datasets in a common coordinate system based on said coordinate datasets; and
tracking said position and orientation of said anatomical feature based on said registering.

14. The method of claim 13 wherein said registering includes generating an anatomical feature model of said anatomical feature based at least on said imaged echo datasets, and registering said anatomical feature model in said coordinate system based on said coordinate datasets.

15. The method of claim 14 wherein said generating said anatomical feature model includes accessing a reference model base, said generating said anatomical feature model being further based on said reference model base.

16. The method of claim 15 wherein said reference model base is selected from a group consisting of: a tibia model base, a femur model base, a spine model base, a shoulder joint model base, a humerus model base, a scapula model base, a forearm model base, a pelvis model base, and an elbow joint model base.

17. The method of claim 13 wherein said emitting phased-array ultrasound signals towards different portions of said anatomical feature includes at least one of steering and focusing said phased-array ultrasound signals towards said different portions of said anatomical feature.

18. The method of claim 13 wherein said tracking includes optically tracking a reference tracker mounted to said ultrasound imaging system during said measuring.

19. The method of claim 13 wherein said tracking includes sensing a relative movement between said ultrasound imaging system and a frame being fixed relative to said anatomical feature and to which said ultrasound imaging system is movable attached.

20. The method of claim 13 further sandwiching a solid acoustically transmissive material between said ultrasound imaging system and an outer-skin surface proximate to said anatomical feature, said solid acoustically transmissive material being provided in the form of a wearable element.

21. The method of claim 13 wherein said imaged echo datasets have corresponding time stamps identifying at what moment in time the echo signals they represent have been measured, said registering being further based on said time stamps.
